(19) United States
(12) Patent Application Publication
(10) Pub. No.: US 2019/0185910 A1
(43) Pub. Date: Jun. 20, 2019

(54) MULTIPLEX PCR METHODS FOR DETECTING GENE FUSIONS, KITS AND COMPOSITIONS

(71) Applicants: Jingfeng Li, New Albany, OH (US); Xiaomin Guo, New Albany, OH (US)

(72) Inventors: Jingfeng Li, New Albany, OH (US); Xiaomin Guo, New Albany, OH (US)

(21) Appl. No.: 16/225,118

(22) Filed: Dec. 19, 2018

Related U.S. Application Data
(60) Provisional application No. 62/607,739, filed on Dec. 19, 2017.

(51) Int. Cl. C12Q 1/686; C12Q 1/6883

(52) U.S. Cl. CPC C12Q 1/686; C12Q 1/6883; C12Q 2600/16; C12Q 2600/118; C12Q 2600/156; C12Q 2600/106

(57) ABSTRACT

Methods for detecting presence or absence of at least two known gene fusions in isolated genomic DNA comprise subjecting isolated genomic DNA to multiplex PCR. For each known gene fusion, the multiplex PCR employs one or a plurality of forward primers which hybridize to a first gene adjacent its fusion breakpoint location, and one or a plurality of reverse primers which hybridize to a second gene adjacent its fusion breakpoint location. The primers hybridize to the respective gene at consecutive respective positions separated from one another by a plurality of base pairs. Amplified products are detected and respectively represent the presence of a gene fusion. Amplified products may be Sanger sequenced to determine the fusion breakpoints. The identified specific fusion is monitored by a designed fusion PCR. Drug-resistant mutations are detected using multiplex mutation real-time PCR in patient plasma cell-free DNA during targeted therapy, for example, tyrosine kinase inhibitor-targeted therapy.

The Sequence Listing entitled “Non-provisional_ST25.txt”, submitted herewith, created Dec. 7, 2018 and having a size of 40,009 bytes, is incorporated herein by reference.

### FIELD OF THE INVENTION

The present invention is directed methods for detecting the presence or absence of one or more known gene fusions in a tissue sample, methods for monitoring detected gene fusions, methods for detecting acquired drug resistant mutations in a plasma sample, and kits and compositions for such methods. The methods, kits and compositions are advantageous for assisting in selection of a suitable therapy for an individual patient, for example, a cancer patient.

### BACKGROUND OF THE INVENTION

The development of targeted therapies based on one or more genetic alterations in a patient are increasing. Accordingly, there is a significant need for efficient and reliable methods for screening individual patients for genetic alterations.

Lung cancer is a leading cause of cancer-related mortality in the United States. Approximately 85% of lung cancers are non-small cell lung cancers (NSCLC), consisting mainly of squamous cell, adenocarcinoma, adenosquamous carcinoma, and large-cell anaplastic carcinoma, with most being adenocarcinomas. Most NSCLCs are diagnosed at an advanced stage, are clinically aggressive, and have a high metastatic potential. Additionally, current NSCLC chemotherapeutic regimens have low efficacy. For example, patients with untreated advanced NSCLC have a median survival of 7-15 months, while those treated with current platinum-based doubled chemotherapy regimens have an 8-12 month median survival. Research into the mechanisms of carcinogenesis and malignant progression of NSCLC has revealed that different driver mutations are altered in this human malignancy, and targeted therapies based on certain genetic alterations in NSCLC tumors have been developed. Identifying mutations in oncogenes associated with non-squamous NSCLC can help determine which patients are more likely to benefit from a targeted therapy. Such oncogenes include EGFR, KRAS, BRAF, PIK3CA, ROS1 and ALK, and molecular diagnostic testing for ALK, ROS1 and EGFR mutations is now recommended for NSCLCs to guide therapy.

Fusion between echinoderm microtubule-associated protein like 4 (EML4) and ALK is seen in approximately 2-7% of patients with NSCLC adenocarcinomas. This and other ALK gene fusions are more common in nonsmokers or light smokers and in those with adenocarcinomas. Because EGFR, ROS1 and ALK mutations are mutually exclusive, patients with ALK rearrangements (gene fusion) are not thought to benefit from treatments targeting the other mutations. For example, EGFR-targeting tyrosine kinase inhibitors (TKIs). Instead, treatment with an ALK inhibitors such as crizotinib (Xalkori), ceritinib (Zykadia), or brigatinib (Alunbrig), are indicated. In patients who received crizotinib as second-line therapy, the 1-year overall survival rate was 70% and the 2-year overall survival rate was 55%. By contrast, ALK-positive matched controls had a 1-year survival of 44% and a 2-year survival of 12%, whereas ALK-negative controls had a 1-year survival of 47% and a 2-year survival of 32%. These data suggest that the presence of the ALK gene fusion itself does not confer a poorer outcome but that the use of crizotinib in ALK-positive patients can improve outcomes. Kwak et al, Anaplastic lymphoma kinase inhibition in non-small-cell lung cancer. N Engl J Med., 363(18):1693-703 (2010); Crinò et al, Initial phase II results with crizotinib in advanced ALK-positive non-small cell lung cancer (NSCLC): PROFILE 1005, J Clin Oncol., 29(suppl 15):Abstract 7514 (2011); Shaw et al, Ceritinib in ALK-rearranged non-small-cell lung cancer, N Engl J Med., 370(13):1189-97 (2014). Based on such data, testing for ALK rearrangement is recommended in patients with metastatic NSCLC adenocarcinoma, and the ALK inhibitor crizotinib is recommended for ALK-gene fusion positive patients.

Similarly, proto-oncogene tyrosine-protein kinase ROS (ROS1) is an orphan receptor tyrosine kinase (RTK) that forms fusions and defines another clinically actionable oncogenic driver mutation in NSCLC. It has been recently reported that approximately 1.4% of NSCLCs harbor ROS1 rearrangements. Of the ROS1 fusion-positive tumors, 30% are known to harbor a recurrent translocation t[5;6][q32;q22], which creates the CD74 molecule, major histocompatibility complex, class II invariant chain (CD74)-ROS fusion kinase. ROS1 is evolutionarily related to ALK, and ALK inhibitors can also be used on ROS1-fusion positive cancers according to their similarity.

Somatic gain-of-function RET mutations have been observed in 30-50% of sporadic medullary thyroid cancer, and somatic RET gene fusions have been observed in 30-50% of sporadic papillary thyroid cancer. The US Food and Drug Administration (FDA) has approved two inhibitory drugs, vandetanib (ZD6474) and cabozantinib (XL184), for the treatment of advanced medullary thyroid cancer. The RET fusions are present in 1-2% of NSCLC adenocarcinoma of patients of both Asian and European descent. Several studies indicate that RET fusion occurs preferentially in young non-smoker and light-smoker patients.

These examples demonstrate the value in the ability to determine if an individual harbors one or more gene fusions which influence the effectiveness of specific therapeutic treatments, particularly in treatment of cancers such as NSCLC. Fluorescent in situ hybridization (FISH) is currently the reference detection method for ALK fusions. This technique uses two specific DNA probes, each coupled to a fluorescent marker, one green and one red, which cover the 2p23 ALK region. In the wild-type scenario, the red signal (3′ ALK) and the green signal (5′ ALK) are adjacent. When the distance between these two signals is more than twice the signal diameter, they are considered separated, reflecting a physical separation of the two DNA regions and hence a translocation (gene fusion).

FISH is considered to be positive for a translocation if >15% of the tumor cells counted in four fields show either a separation between the green and red signals or a single red signal with loss of the associated green signal. This 15% threshold allows for errors due to background noise, reading or aberrant hybridization. At least 50 cells must be counted, with a second count of another 50 cells by a second reader if there are between 10% and 50% positive cells. The strengths of FISH lie in its ability to detect ALK rearrangements irrespective of the variant or the fusion protein, as well as its correlation with clinical efficacy. It has been approved by the FDA for use in connection with crizotinib therapy (Vysis ALK Break-Apart FISH Probe kit; Abbott Molecular, Inc., Des Plaines, Ill., USA). However, the use of FISH analysis for detecting ALK translocations can be challenging as 1) the technique is relatively expensive, 2) accurate interpretation of the results requires the expertise and experience of a trained cytologist who must view testing of multiple tissue sections, 3) the technique does not identify specific translocation types, and 4) the technique often has a lengthy turn-around time.

Immunohistochemistry (IHC) is another method for detecting ALK rearrangements in lung cancer. Initially, IHC encountered sensitivity issues and occasional false-positive results. However, newer ultrasensitive IHC techniques appear to offer a more reliable and sensitive screening method. The positivity threshold is typically visual, requiring moderate to intense staining in 5-10% of cells. Advantages of IHC are mainly its low cost in terms of both time and manpower, but standardization of the test is difficult. The challenges in developing IHC for ALK detection in NSCLC are: 1) tissue preparation, 2) antibody choice, 3) signal enhancement systems, and 4) the optimal scoring system. While IHC is a reliable screening tool, FISH confirmation is required in the event of positive IHC and even in some cases for negative IHC in patients presenting predictive rearrangement markers, including younger age, light smokers (10 pack-years) and testing negative for other mutations, notably EGFR and KRAS.

Reverse transcriptase polymerase chain reaction (RT-PCR) and quantitative RT-PCR (qRT-PCR) are also used as diagnostic techniques for ALK translocations. Typically, RNA is converted into cDNA by reverse transcriptase and the cDNA is PCR amplified with specific primers. See, for example, Sanders et al, U.S. Pat. No. 9,175,350 B2, and Begovich et al, US 2016/0304937 A1. Amplification requires primer sets specific for each translocation. This highly specific technique offers the additional advantage of identifying the fusion gene associated with ALK. Its limited use to date is due to the requirement of a quality DNA sample from a formalin-fixed, paraffin-embedded (FFPE) tissue sample or from fresh or frozen tumor tissue. Additionally, detection of fusion genes by RT-PCR or qRT-PCR might not be successful because of the highly variable nature of gene fusions.

Accordingly, a need exists for a simple, high-throughput method for detecting gene fusions, including, but not limited to, those involving ALK, ROS1, RET1 and NRTK1 translocations. Detection of translocations in accordance with the methods of the invention is useful for, inter alia, targeted therapy.

### SUMMARY OF THE INVENTION

Accordingly, an object of the present invention is to provide a simple, high-throughput method for detecting gene fusions, including, but not limited to, those involving ALK, ROS1, RET and/or NRTK1 translocations.

In one embodiment, the invention is directed to a method for detecting the presence or absence of at least two known gene fusions in a tissue sample, wherein each gene fusion is formed between a respective first gene at a first fusion breakpoint location and a respective second gene at a second fusion breakpoint location. The method comprises (a) providing genomic DNA isolated from the tissue sample, (b) subjecting the isolated genomic DNA to multiplex PCR, wherein, for each known gene fusion, the multiplex PCR employs one or a plurality of forward primers which hybridize to the respective first gene adjacent the first fusion breakpoint location, wherein the plurality of forward primers hybridize to the respective first gene at consecutive respective positions along the first gene and are separated from one another by a first plurality of base pairs, and one or a plurality of reverse primers which hybridize to the respective second gene adjacent the second fusion breakpoint location, wherein the plurality of reverse primers hybridize to the respective second gene at consecutive respective positions along the second gene and are separated from one another by a second plurality of base pairs, and (c) detecting if one or more amplified products are formed, each amplified product respectively representing the presence of a gene fusion at a genomic level.

In another embodiment, the invention is directed to a method for identifying a gene fusion in a tissue sample when, in a method as described is conducted to determine the presence or absence of at least two known gene fusions in the tissue sample and at least one amplified product has been detected in step (c) of the method, (d) subjecting genomic DNA isolated from the tissue sample of the individual to PCR for each gene fusion for which forward primer(s) and reverse primer(s) were employed in step (b), the individual PCR employing the forward primer(s) and the reverse primer(s) employed in the multiplex PCR for the respective gene fusion; and (e) in each individual PCR, detecting if an amplified product is formed, the amplified product representing the presence of the respective gene fusion.

In a further embodiment, the invention is directed to a method for detecting the presence or absence of a known gene fusion in a tissue sample, wherein the gene fusion is formed between a first gene at a first fusion breakpoint location and a second gene at a second fusion breakpoint location. The method comprises (a) providing genomic DNA isolated from the tissue sample, (b) subjecting the isolated genomic DNA to PCR amplification, the PCR amplification employing one or a plurality of forward primers which hybridize to the respective first gene adjacent the first fusion breakpoint location, wherein the plurality of forward primers hybridize to the respective first gene at consecutive respective positions along the first gene and are separated from one another by a first plurality of base pairs, and one or a plurality of reverse primers which hybridize to the respective second gene adjacent the second fusion breakpoint location, wherein the plurality of reverse primers hybridize to the respective second gene at consecutive respective positions along the second gene and are separated from one another by a second plurality of base pairs, and (c) detecting if an amplified product is formed, the amplified product representing the presence of the gene fusion.

In specific embodiments, the methods may be used to detect acquired drug resistant mutations in a plasma sample.

In certain embodiments, the invention further relates to methods of monitoring a detected gene fusion. For example, the methods may be used to facilitate monitoring treatment in a patient, wherein an amplified product is subjected to Sanger sequencing, for example, using multiplex PCR primers. Forward and reverse primers are designed around the gene fusion in the amplified product, operable to amplify a fragment encompassing the gene fusion. The primers are then used to monitor the amount of fusion product in patient plasma cell-free DNA (cfDNA), for example, to assess cancer progression or cancer regression during targeted treatment.

In additional embodiments, the invention is directed to a method for monitoring a patient undergoing targeted therapy with a drug to which resistance may develop. The method comprises detecting the presence or absence of one or more acquired functional mutations associated with the drug resistance in a tissue sample from the patient according to the methods described above. In a more specific embodiment, the invention is directed to a method for monitoring a patient undergoing targeted therapy with crizotinib, and the method comprises detecting ALK- and ROS1-crizotinib resistant mutations using a method as described herein employing multiplex real-time PCR. Detection of drug resistant mutations guides second or third line targeting treatment.

In yet a further embodiment, the invention is directed to kits for detecting the presence or absence of a gene fusion in an isolated genomic DNA sample, which kit contains a select combination of reverse primers and forward primers. The invention is also directed to compositions comprising an isolated DNA sample from a patient non-small cell lung cancer tumor and a select combination of reverse primers and forward primers.

The present methods, kits and compositions provide a simple and high throughput method for detecting the presence or absence of known gene fusions in a tissue sample. As the presence or absence such gene fusions can be a significant factor in selecting a treatment therapy for disease such as cancer, and, as demonstrated above, for selecting a treatment therapy for NSCLC, the present methods provide a significant step forward in targeting patient therapies for combating such diseases. Additionally, the present methods open additional alternatives for monitoring disease progress and/or treatment therapy progress, which cannot be accomplished by other methods. These and additional advantages of the present invention will be more fully apparent and understood in view of the following detailed description.

### BRIEF DESCRIPTION OF THE DRAWINGS

The following detailed description of the invention may be more fully understood in view of the drawings, in which:

FIG. 1 presents a schematic representation of a first embodiment of a method of the invention.

FIG. 2 presents a schematic representation of a specific embodiment of a method of the invention described in Example 1.

FIG. 3 presents electrophoresis gels resulting from the multiplex PCRs described in Example 2.

FIG. 4 presents an electrophoresis gel resulting from the multiplex PCR described in Example 3.

FIG. 5 presents electrophoresis gels resulting from the multiplex PCRs described in Example 4.

FIG. 6 presents a schematic representation of competitive mutation-specific SYBR® real-time PCR for detection of acquired crizotinib-resistant ALK mutations as described in the specification.

FIG. 7 shows the results of SYBR® real-time PCR detection of ALK resistant-mutations L1196M and G1269A as described in Example 5.

The drawings will be more fully understood in view of the following detailed description and the Examples presented below.

### DETAILED DESCRIPTION

The present invention provides a multiplex polymerase chain reaction (PCR)-based method for detecting the presence or absence of a known gene fusion, differing from the wild-type polynucleotide sequence of a gene, using isolated genomic DNA. The DNA is isolated from a sample from an individual, i.e., a clinical tumor biopsy, a formalin-fixed, paraffin-embedded (FFPE) tissue sample, or a fresh frozen tissue sample. Genomic DNA can also be isolated from patient blood (plasma) DNA to detect one or more translocations in blood disease, i.e., BCR-ABL with chronic myelogenous leukemia (CML).

In certain embodiments, the sample is a plasma sample or, more specifically, a cell-free plasma DNA sample (plasma cfDNA). In certain embodiments, plasma cfDNA may be subjected to an enrichment process for enrichment of mutant alleles. For example, to increase the test sensitivity of rare mutations in plasma cfDNA, the mutated alleles in the cfDNA are enriched using any one or more of known enrichment methods, e.g. MutS/L enzyme binding, or preferred PCR amplification of mutant alleles. In one embodiment, COLD-PCR (co-amplification at lower denaturation temperature-PCR) is used. COLD-PCR is a modified PCR protocol that enriches both known and unknown minority alleles irrespective of mutation type and position. The ability to preferentially amplify low-level DNA mutations in the presence of excess wildtype alleles is useful for the detection of mutations. Mutant alleles are enriched by 10-100 fold by COLD-PCR. In a specific embodiment, COLD-PCR is performed on all tested mutant locus to enrich mutant alleles simultaneously, and the mutation enriched samples are then subjected to the described mutation-specific real-time PCR. The combination of these steps allows detection of as low as 0.01% (one copy mutant in 10,000 copy wild-type) mutations in a sample.

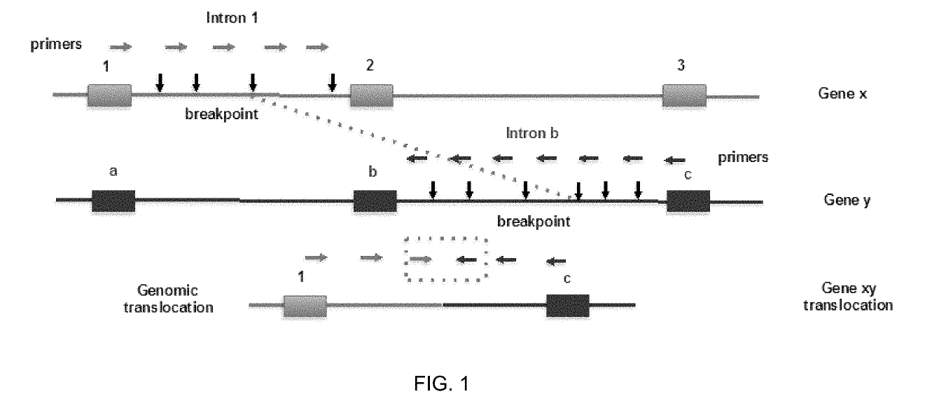

The principle of a first embodiment of the invention is shown schematically in FIG. 1. More specifically, FIG. 1 shows a known gene X exon 1 and gene Y exon c fusion due to translocation. A breakpoint could be located at any position in gene X intron 1, indicated by vertical black arrows, and in any position in gene Y intron b, indicated by vertical black arrows. Primers are designed to hybridize at intervals, i.e., consecutive respective positions, of, for example, every about 0.1 to about 4 kb, from about 0.25 to about 3 kb, from about 0.5 to about 2 kb, or from about 0.5 to about 1 kb, along the respective genes adjacent the fusion breakpoint locations, i.e., the breaking introns, as indicated by horizontal grey arrows in intron 1 (forward primers) and horizontal black arrows in intron b (reverse primers). As shown in FIG. 1, the forward and reverse primers hybridizing closest to the breakpoints forming the gene fusion allow PCR amplification of the genomic translocation by the forward primer from intron 1 and the reverse primer from intron b, indicated in FIG. 1 by the primers in the dashed rectangle. Thus, the closest flanking primers will pair to amplify the sequence spanning the translocation sites. By detecting an amplified product in the PCR, the presence of a gene fusion is confirmed. The method therefore allows the determination of the presence (or absence) of a gene fusion using primers from different known translocated genes together to detect a gene fusion by real time PCR in a single tube.

More generally, in one embodiment, the method of the invention allows detection of the presence or absence of at least two known gene fusions in a tissue sample. In specific embodiments, the methods may be used to detect the presence or absence of at least three, four, five, six, seven, eight or more, known gene fusions in a tissue sample, wherein each gene fusion is formed between a respective first gene at a first fusion breakpoint location and a respective second gene at a second fusion breakpoint location. Generally, a fusion breakpoint location refers to the intron at which fusion occurs in the respective gene. The method comprises (a) providing genomic DNA isolated from the tissue sample, and (b) subjecting the isolated genomic DNA to multiplex PCR. In the multiplex PCR, for each of at least two, three, four, five, six, seven, eight or more, known gene fusions, the multiplex PCR employs one or a plurality of forward primers which hybridize to the respective first gene adjacent the first fusion breakpoint location, wherein the plurality of forward primers hybridize to the respective first gene at consecutive respective positions along the first gene and are separated from one another by a first plurality of base pairs, and one or a plurality of reverse primers which hybridize to the respective second gene adjacent the second fusion breakpoint location, wherein the plurality of reverse primers hybridize to the respective second gene at consecutive respective positions along the second gene and are separated from one another by a second plurality of base pairs. The number of primers, i.e., one or a plurality, for each gene of a specific known gene fusion will depend on the length of the introns at which the fusion occurs. Additionally, for a fusion breakpoint which is close to an exon, the one or plurality of primers may hybridize at least in part along the adjacent exon. For a small intron, i.e., less than 1 kb, a primer located in the nearby exon would cover all breakpoints in the intron. When a plurality of primers are employed and the primers are designed to hybridize at intervals, i.e., consecutive respective positions, along the respective gene, the respective positions are separated by about 0.1 to about 4 kb, by about 0.25 to about 3 kb, by about 0.5 to about 2 kb, or by about 0.5 to about 1 kb, along the respective genes adjacent the fusion breakpoint locations. The respective primers for each gene fusion may then be provided in a single PCR reaction setting, i.e., a single test tube. The detection of one or more amplified products resulting from the PCR indicates the presence of at least one gene fusion in the tissue sample and each amplified product respectively represents the presence of a gene fusion.

Due to the specificity of translocation amplification in a sample, the multiplex PCR methods of the invention are particularly advantageous in that they allow for identification and characterization of translocations, even against a background containing a vast excess of wild-type molecule. For example, the methods allow for detection of chromosomal translocations and mapping their breakpoints in samples of genomic DNA containing up to 99% of wild-type DNA contamination.

In a further embodiment of the invention, when, in a method as described is conducted to determine the presence or absence of at least two known gene fusions in the tissue sample and at least one amplified product has been detected in step (c) of the method, a specific gene fusion in the tissue sample can be identified by (d) subjecting genomic DNA isolated from the tissue sample of the individual to PCR for each gene fusion for which forward primer(s) and reverse primer(s) were employed in step (b), the respective individual PCRs employing the forward primer(s) and the reverse primer(s) employed in the multiplex PCR for the respective gene fusion; and (e) in each individual PCR, detecting if an amplified product is formed, the amplified product representing the presence of the respective gene fusion.

While the advantages of reduced testing time, effort and cost provided by the present methods are increased when the multiplex PCR is conducted for an increased number of known gene fusions, the method of the invention may also be used to detect the presence or absence of a single gene fusion. Thus, in one embodiment, the method comprises detecting the presence or absence of a single known gene fusion in a tissue sample, wherein the gene fusion is formed between a first gene at a first fusion breakpoint location and a second gene at a second fusion breakpoint location. The method comprises (a) providing genomic DNA isolated from the tissue sample, (b) subjecting the isolated genomic DNA to PCR amplification, the PCR amplification employing one or a plurality of forward primers which hybridize to the respective first gene adjacent the first fusion breakpoint location, wherein the plurality of forward primers hybridize to the respective first gene at consecutive respective positions along the first gene and are separated from one another by a first plurality of base pairs, and one or a plurality of reverse primers which hybridize to the respective second gene adjacent the second fusion breakpoint location, wherein the plurality of reverse primers hybridize to the respective second gene at consecutive respective positions along the second gene and are separated from one another by a second plurality of base pairs, and (c) detecting if an amplified product is formed, the amplified product representing the presence of the gene fusion.

In additional embodiments of the methods of the invention, an amplified product, for example, from an individual PCR, may be sequenced using any technique known in the art. An exemplary sequencing method is Sanger sequencing, but other sequencing techniques known in the art are applicable. From the sequenced amplified product, a forward primer and a reverse primer around the gene fusion in the amplified product can be designed in order to amplify a gene fragment encompassing the gene fusion. A suitable fragment may have a length of from about 50 to about 150 base pairs, from about 75 to about 125 base pairs, or about 100 base pairs. The primer pair may then be used to monitor disease progress, for example, cancer progression or regression, therapeutic treatment, or the like, in a noninvasive manner. For example, the so-designed primer pair may then be used for PCR-based specific translocation detection in isolated DNA from cell-free plasma from a patient's blood sample as a noninvasive translocation monitoring during a treatment process.

In specific embodiments, when a patient is undergoing treatment with a drug which is known to generate drug resistance over time owing to one or more acquired functional mutations in a fusion gene, the patient can be monitored over the course of the treatment in order to allow early detection of the fusion gene mutation(s) and revision of the patient therapy, for example to begin treatment with a second or third line treatment drug. Such methods are particularly valuable in various cancer treatment where first line treatment drugs are known to generate drug resistance over time. For example, patients undergoing first-line treatment with crizotinib against ALK and ROS1 translocations will typically acquire drug resistance in about one year. In more than 50% of the patients, the acquired resistance is due to ALK and/or ROS1 kinase domain mutations, which could be targeted by second and third generation TKI inhibitors, i.e. ceritinib and lorlatinib to limit tumor development. Accordingly, early detection of crizotinib-resistant mutations in a patient's plasma cfDNA is a significant advance. In a specific embodiment, the method comprises detecting one or more crizotinib-resistant ALK mutations selected from 1511Tins, L1152R, C1156Y, 11171T/N/S, F1174C/L/V, L1196M, L1198F, G1202R, S1206Y and G1269A, and combinations thereof. In another specific embodiment, the method comprises detecting one or more crizotinib-resistant ROS1 mutations selected from L2026M, G2032R, D2032N, L2155S, S1986Y and S1986F, and combinations thereof. Importantly, this type of monitoring cannot be done by currently available fusion detection methods, i.e., IHC, FISH and real time RT-PCR. Monitoring using a method of the present invention may be conducted, for example, at the initiation of treatment and periodically thereafter, i.e., weekly, biweekly, monthly, bimonthly, or otherwise as appropriate.

In a specific embodiment therefore, the methods of the invention may be employed to detect the presence or absence of an ALK gene fusion in a tissue sample. Cancers associated with an ALK fusion are very sensitive to ALK inhibitors such as crizotinib and ceritinib. The efficient and powerful testing for ALK gene fusions provided by the present methods represent a significant and valuable opportunity for selecting a patient-targeted treatment. EML4-ALK is the most dominant gene fusion found in NSCLC. The KIF5B, KLC1, TFG, SEC31A, TPR, SQSTM1, DCTN1, STRN, PPFIBP1 and HIP1 genes can also fuse with ALK in a similar manner in NSCLC. Accordingly, in a method according to the present invention, multiplex PCR is conducted to detect the presence or absence of two, up to all, of the known gene fusions of ALK translocations in NSCLC. In a specific embodiment, the multiplex PCR employs four primers designed to hybridize at ALK intron 19 (about 2 kb) and one primer designed to hybridize at ALK exon 20 (about 200 bp). The distance between two consecutive primers on intron 19 is about 0.5-1 kb and all primers are designed at the opposite orientation to ALK gene transcription direction, i.e., as reverse primers. The multiplex PCR further employs primers designed to hybridize, for example, to EML4 at exon 13 and intron 13, exon 16 and intron 16, exon 20 and intron 20, exon 14 and intron 14, exon 15, exon 2 and intron 2, and/or exon 17 and intron 17, to KIF5B at exon 24 and intron 24, exon 17 and intron 17, and/or exon 15 and intron 15, to KLC at exon 9 and intron 9, to TFG at intron 2, and/or intron 3, to SEC31A at exon 21 and intron 21, to TPR at exon 15 and intron 15, to SQSTM1 at exon 5 and intron 5, to DCTN1 at exon 26 and intron 26, to STRN at exon 3 and intron 3), to PPF1BP1 at exon 8 and intron 8, and/or exon 12 and intron 12, and/or to HIP1 at exon 21, exon 28, and/or exon 30. In a specific embodiment of the present methods to determine the presence or absence of two or more gene fusions out of all of these known gene fusions, a total of about 150 primers are designed in the indicated partner genes exons and introns, with a distance around 0.5-1 kb, at the same orientation as the gene transcription direction, i.e., as forward primers. In a specific embodiment, these forward primers are divided into two groups based on PCR primer optimization for maximum amplification efficiency, and each group is mixed together with, for example, the indicated five ALK mixed primers for three multiplex real time PCR to detect all known ALK translocations in tumor samples.

Thus, in an additional embodiment, the invention is directed to a kit for detecting the presence or absence of at least one ALK gene fusion in a tissue sample. In a more specific embodiment, the kit comprises a plurality of reverse primers designed to hybridize at ALK intron 19 and ALK exon 20, with a distance around 0.5-1 kb between primers, and a plurality of forward primers designed to hybridize to EML4 at exon 13 and intron 13, exon 16 and intron 16, exon 20 and intron 20, exon 14 and intron 14, exon 15, exon 2 and intron 2, and/or exon 17 and intron 17, to KIF5B at exon 24 and intron 24, exon 17 and intron 17, and/or exon 15 and intron 15, to KLC at exon 9 and intron 9, to TFG at intron 2, and/or intron 3, to SEC31A at exon 21 and intron 21, to TPR at exon 15 and intron 15, to SQSTM1 at exon 5 and intron 5, to DCTN1 at exon 26 and intron 26, to STRN at exon 3 and intron 3), to PPF1BP1 at exon 8 and intron 8, and/or exon 12 and intron 12, and/or to HIP1 at exon 21, exon 28, and/or exon 30, with a distance around 0.5-1 kb between consecutive primers hybridizing at the same or adjacent introns and/or exons. In a more specific embodiment, the kit comprises a plurality of forward primers designed to hybridize to all of the noted introns and exons.

In another specific embodiment, the methods of the invention may be employed to detect the presence or absence of a ROS1 gene fusion in a tissue sample. ALK inhibitors can also be used on ROS1-fusion positive patients with increased efficacy. Accordingly, in a method according to the present invention, multiplex PCR is conducted to detect the presence or absence of at least two, up to all, of the known gene fusions of ROS1 translocations in NSCLC. In a specific embodiment, the multiplex PCR employs eleven primers designed to hybridize to ROS1 at exon 32 and intron 31, four primers are designed to hybridize to ROS1 at exon 34 and intron 33, and five primers designed to hybridize to ROS1 at exon 35 and intron 34. The distance between two neighbor primers is about 0.5-1 kb and all primers are designed at opposite orientation to ROS1 gene direction, i.e., as reverse primers. These eight primers are mixed together for use in a single multiplex. The multiplex PCR further employs primers designed to hybridize, for example, to slc34a2 at exon 4 and intron 4, and/or exon 12 and intron 12, to SDC4 at exon 2 and intron 2; and/or exon 4 and intron 4, to CD74 at exon 6 and intron 6, to EZR at exon 10 and intron 10, to LRIG3 at exon 16 and intron 16, to TPM3 at exon 2 and intron 2 and/or exon 8 and intron 8, to GOPC at exon 8 and intron 8, and/or to CCDC6 at exon 6 and intron 6. In a specific embodiment of the present methods to determine the presence or absence of at least one gene fusion out of all of these known gene fusions, a total of about 35 primers are designed in the indicated partner genes exons and introns, with a distance of about 0.5-1 kb, at the same orientation as the gene transcription direction, i.e., as forward primers. These primers are mixed together for one multiplex real time PCR by pairing with the noted 20 ROS1 primers to detect all known ROS1 translocations in tumor samples.

Thus, in an additional embodiment, the invention is directed to a kit for detecting the presence or absence of at least one ROS1 gene fusion in a tissue sample. In a more specific embodiment, the kit comprises a plurality of reverse primers designed to hybridize to ROS1 at exon 32 and intron 31, exon 34 and intron 33, and exon 35 and intron 34, with a distance around 0.5-1 kb between consecutive primers hybridizing at the same or adjacent introns and/or exons, and a plurality of forward primers designed to hybridize to slc34a2 at exon 4 and intron 4, and/or exon 12 and intron 12, to SDC4 at exon 2 and intron 2; and/or exon 4 and intron 4, to CD74 at exon 6 and intron 6, to EZR at exon 10 and intron 10, to LRIG3 at exon 16 and intron 16, to TPM3 at exon 2 and intron 2 and/or exon 8 and intron 8, to GOPC at exon 8 and intron 8, and/or to CCDC6 at exon 6 and intron 6, with a distance around 0.5-1 kb between consecutive primers hybridizing at the same or adjacent introns and/or exons. In a more specific embodiment, the kit comprises a plurality of forward primers designed to hybridize to all of the noted introns and exons.

In another specific embodiment, the methods of the invention may be employed to detect the presence or absence of at least one of known RET gene fusions in a tissue sample. Targeted therapy may be selected for RET-fusion positive patients. In a specific embodiment, the methods of the invention employ multiplex PCR to detect the presence of all known RET translocations in NSCLC. In a specific embodiment, the multiplex PCR employs four primers designed to hybridize to RET at exon 12 and intron 11, two primers designed to hybridize to RET at exon11 and intron 10, and two primers designed to hybridize to RET at exon 8 and intron 7. The distance between two neighbor primers is 0.5-1 kb and all primers are designed at opposite orientation to the RET gene transcription direction, i.e., as reverse primers. These eight primers are mixed together. The multiplex PCR further employs primers designed to hybridize, for example, to KIF5B at exon 15 and intron 15, exon 16 and intron 16, exon 22 and intron 22, exon 23 and intron 23; and/or exon 24 and intron 24, to TRIM33 at exon 14 and intron 14, to NCOA4 at exon 6 and intron 6, and/or to CUX1 at exon 19 and intron 19. In a specific embodiment of the present methods to determine the presence or absence of at least one gene fusion out of all of these known gene fusions, a total 35 primers are designed in the indicated partner genes exons and introns with a distance around 0.5-1 kb and at same orientation as gene transcription direction, i.e., as forward primers. In a specific embodiment, these forward primers are divided in two groups based on PCR primer optimization for maximum amplification efficiency, mixed together for two multiplex real time PCR by pairing with the eight RET mixed primers to detect all known RET translocations in tumor samples.

Thus, in an additional embodiment, the invention is directed to a kit for detecting the presence or absence of at least one RET gene fusion in a tissue sample. In a more specific embodiment, the kit comprises a plurality of reverse primers designed to hybridize to RET at exon 12 and intron 11, exon11 and intron 10, and exon 8 and intron 7, with a distance around 0.5-1 kb between consecutive primers hybridizing at the same or adjacent introns and/or exons, and a plurality of forward primers designed to hybridize to KIF5B at exon 15 and intron 15, exon 16 and intron 16, exon 22 and intron 22, exon 23 and intron 23; and/or exon 24 and intron 24, to TRIM33 at exon 14 and intron 14, to NCOA4 at exon 6 and intron 6, and/or to CUX1 at exon 19 and intron 19, with a distance around 0.5-1 kb between consecutive primers hybridizing at the same or adjacent introns and/or exons. In a more specific embodiment, the kit comprises a plurality of forward primers designed to hybridize to all of the noted introns and exons.

In a specific embodiment, five multiplex PCRs may be conducted (two for ALK gene fusions, one for ROS1 gene fusions and 2 for RET gene fusions as described above) to detect all known ALK, ROS1 and RET translocations in NSCLC, which consists over 10% of mutations from tumor samples.

As will be apparent from the description of the methods and kits herein, the methods and/or kits for detecting the presence or absence of a plurality, i.e., at least three, four, five, six, seven, eight, or more gene fusions, may employ two or more multiplex PCR according to step (c), wherein the primers for the respective known gene fusion are divided among the multiplex PCRs to minimize dimer formation between primers and therefore maximize amplification efficiency. That is, PCR efficiency is mainly affected by self-dimerization and hetero dimerization between primers. Dimer formation is based on primer sequences, so the greater number of primers in a multiplex PCR, the higher the possibility of hetero dimer formation which will limit PCR detection efficiency. Primers having high hetero dimer formation can be physically separated, namely employed in the different multiplex PCR reactions. As an example, the ALK fusion multiplex PCR described herein, in one embodiment, is conducted using three multiplex PCR. Of course, four, five or more multiplex PCR may be conducted with even higher PCR efficiency, although requiring more sample DNA.

Any of the kits according to the invention as described herein may further comprise a negative control isolated DNA sample which is free of the gene fusion(s) of interest, i.e., free of a positive fusion DNA.

According to a further embodiment therefore, the invention is directed to compositions comprising an isolated DNA sample and selected combinations of the reverse and forward primers employed in the multiplex PCR as described herein. In a specific embodiment, the isolated DNA sample is from a patient non-small cell lung cancer tumor. In further embodiments, the reverse primers and forward primers are those of the kits described above or as described in the following Examples.

The multiplex PCR may be conducted according to the techniques known in the art and using polymerase known in the art. In specific embodiments, the polymerase employed in the PCR may be Taq polymerase, or more specifically, platinum Taq polymerase (Invitrogen), but the methods are not limited to the use of such polymerase and one or more other polymerase may be used in the present methods. Optionally, one or more enhancing agents may be employed in the PCR reactions, for example, to improve yield and/or specificity of targets in the PCR amplification. Commonly used PCR enhancing agents include, but are not limited to, betaine and dimethyl sulfoxide (DMSO). In one specific embodiment, 5% DMSO is included in the PCR reaction to increase multiplex PCR efficiency.

The methods of the invention can be used to detect any known oncogene translocation/gene fusion in cancer and are specifically advantageous for use in combination with the selection of targeted treatment therapies. For example, an estimated 1,500-5,000 patients harbor TRK-fusion positive cancers in United States annually. TRK fusion-positive adult and pediatric advanced solid tumors are represented in number of cancer types including salivary gland tumor, other soft tissue sarcoma, infantile fibrosarcoma, thyroid tumor, colon cancer, lung cancer, melanoma, gastrointestinal stromal tumor, cholangiocarcinoma, appendix tumor, breast cancer, and pancreatic cancer. Larotrectinib is the first and only selective pan-TRK inhibitor in clinical development. On Nov. 27, 2018 U.S. FDA approved Larotrectinib under the brand name Vitrakvi® with 75% overall response rate (ORR) for patients with advanced solid tumors harboring an NTRK gene fusion. The methods of the invention can be employed to detect TRK fusion genes in isolated genomic DNA to provide a fast and efficient manner to guide targeted therapy of Larotrectinib. The specific TRK fusions detected by the inventive methods can easily be monitored in individual patients during clinical treatment. The methods of the invention can be employed to detect the presence of TPM4-ALK gene fusion which may be present in esophageal squamous carcinoma, NPM-ALK gene fusion which may be present in anaplastic Non-Hodgkin's lymphoma (ALCL) and ALK and RET translocations in thyroid cancer. Further examples of gene fusions/translocations which may be detected in a fast and efficient manner to guide targeted therapy and/or for monitoring purposes, include, but are not limited to, BCR-ABL1 (chronic myeloid leukemia, CML), RBN15-MKL1 (acute megakaryoblastic leukemia, AML), NPM1-ALK (anaplastic large T-cell lymphoma), IGH-MYC (Burkitt lymphoma/leukemia), RUNX1-RUNX1T1 (acute myeloid leukemia), ETV6-RUNX1 (B-cell precursor acute lymphoblastic leukemia), IGH-MAF (multiple myeloma), PML-RARA (acute promyelocytic leukemia), CD74-NTRK1 (non-small cell lung cancer), MPRIP-NTRK1 (non-small cell lung cancer), FGFR2-KIAA1967 (lung squamous cell carcinoma), FGFR3-TACC3 (various carcinomas), and others, including but not limited to TPM3-NTRK1, LMNA-NTRK1, SQSTM1-NTRK1, TPR-NTRK1, PEAR1-NTRK1, IRF2BP2-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, QKI-NTRK2, NACC2-NTRK2, VCL-NTRK2, AGBL4-NTRK2, PAN3-NTRK2, AFAP1-NTRK2, DAB21P-NTRK2, TRIM24-NTRK2, SQSTM1-NTRK2, ETV6-NTRK3, BTBD1-NTRK3, EML4-NTRK3, TFG-NTRK3, RBPMS-NTRK3, and LYN-NTRK3. The present methods are also suitable for use in detecting gene fusions known to occur in other translocation-harboring malignant solid tumors and sarcomas including, but are not limited to, thyroid cancer, renal cell carcinoma, soft tissue sarcoma, Ewing sarcomas, and the like.

Any suitable method may be used to detect the amplification products. In one embodiment, the detection is accomplished with electrophoresis. In a specific embodiment, the detecting is accomplished using a real-time PCR-based detection system, such as EvaGreen® or SYBR®green. Since the amplified translocation PCR fragment could be up to 2 kb, the Invitrogen platinum Taq polymerase combined with EvaGreen® may be used, for example, to perform multiplex real-time PCR on the ABI StepOne real time PCR system.

In certain embodiments which may be used to facilitate monitoring treatment in a patient, amplified products are subjected to Sanger sequencing, for example, using multiplex PCR primers. Forward and reverse primers are designed around the gene fusion operable to amplify a fragment encompassing the gene fusion. The primers are then used to monitor the amount of fusion product in patient plasma cell-free DNA (cfDNA), for example, during treatment of the patient with a drug to which resistance may develop.

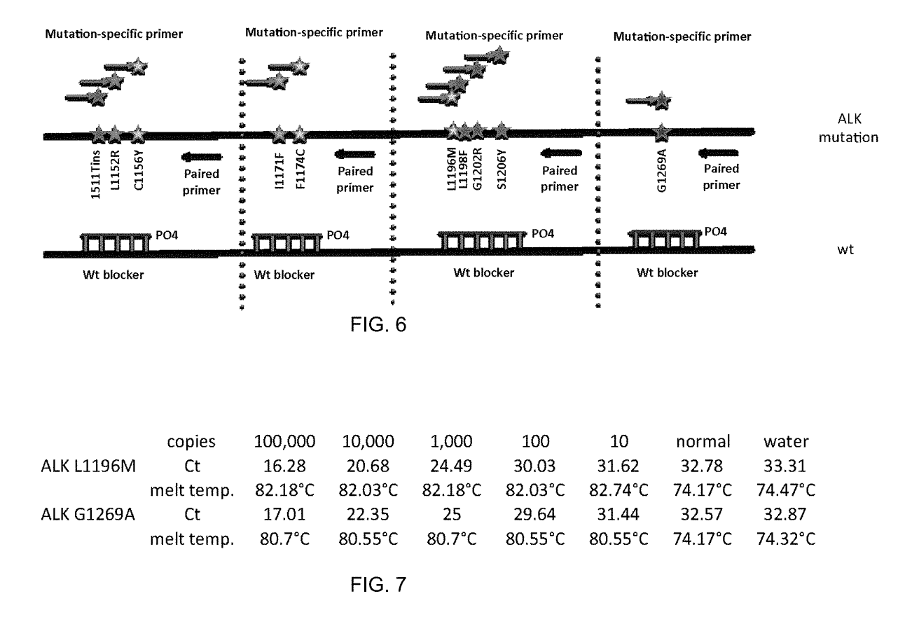

For example, a patient with ALK-positive target therapy with crizotinib will acquire drug resistance within months of treatment. The acquired crizotinib resistance could be due to new oncogenic mutations such as EGFR, KIT or KRAS mutations in tumor, or mostly secondary point mutations in the kinase domain of ALK conferring resistance to this first generation ALK kinase inhibitor. Second and third generation ALK inhibitors, for example, ceritinib and lorlatinib, can overcome crizotinib resistance. Identification of these secondary crizotinib-resistant ALK mutations in patients prior to and during treatment with an ALK inhibitor could enable clinicians to detect the development of resistance well in advance of the detection of disease progression by imaging techniques. The described method of the present invention provides a multiplex competitive mutation detection method based on SYBR® green or Taqman real time PCR to detect 0.1-0.01% crizotinib-resistant ALK mutations of 1511Tins, L1152R, C1156Y, 11171T, I1171S, I1171N, F1174V, F1174C, F1174L, L1196M, L1198F, G1202R, S1206Y and G1269A in plasma cfDNA from crizotinib-resistant ALK positive NSCLC patients. Such a method is shown schematically in FIG. 6, and may be used to determine subsequent treatment decisions. Optionally, mutant alleles maybe enriched from patient plasma cfDNA as described above.

It has been reported that cancers eventually develop crizotinib resistance due to acquired mutations such as L2026M, G2032R, D2032N, L2155S, S1986Y and S1986F in ROS1, and the cMET/RET/VEGFR inhibitor cabozantinib (XL184) and lorlatinib effectively overcome the ROS1 crizotinib-resistant mutations. The present invention provides a multiplex competitive mutation detection method based on SYBR® green or Taqman real time PCR to detect 0.1-0.01% crizotinib-resistant ROS1 mutations of L2026M, G2032R, D2032N, L2155S, S1986Y and S1986F in plasma cfDNA from crizotinib-resistant ROS1 positive NSCLC patients which can shape subsequent treatment decisions. Optionally, mutant alleles maybe enriched from patient plasma cfDNA as described above.

The methods of the invention may be further exemplified in connection with specific gene fusion detection methods as described in the Examples.

### Example 1

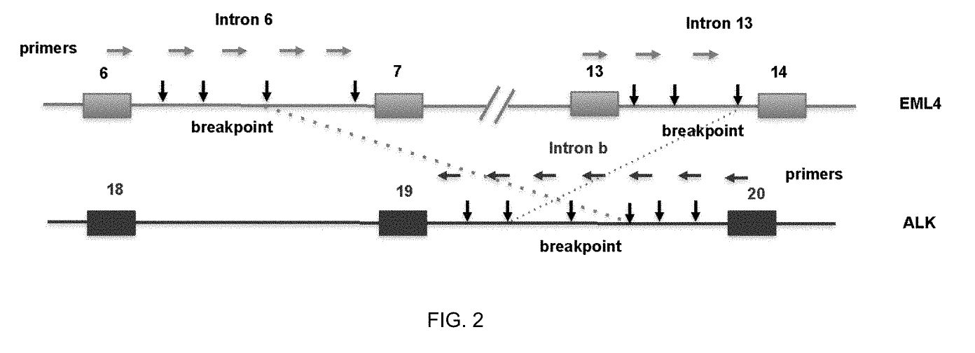

In this example, primers are designed for use in a method according to the invention to detect the presence or absence of gene fusions/translocations between EML4 intron 6 or EML4 intron 13 and ALK intron 19. Specifically, five polynucleotide reverse primers aligned on known translocation ALK exon 20 and intron 19 (SEQ ID NO: 1-5) are designed to hybridize at consecutive respective locations separated from one another at a distance about 0.5-1 kb. According the size of the translocation-involved intron, 10 polynucleotide forward primers aligned on known translocation EML4 exon 13 and intron 13 (SEQ ID NO: 6-15) are designed to hybridize at consecutive respective locations separated from one another at a distance about 0.5-1 kb, and 26 forward primers aligned on known translocation EML4 exon 6 and intron 6 (SEQ ID 16-41) are designed to hybridize at consecutive respective locations separated from one another at a distance about 0.5-1 kb, as shown schematically in FIG. 2. Any translocation between breakpoint in ALK intron 19 and breakpoint in EML4 intron 6 and intron 13 will be detected by a pair of primers near breakpoints in the multiplex PCR.

### Example 2

In this example, fusion products were generated and used as a sample in a multiplex PCR method according to the invention to detect ALK gene fusions.

More specifically, fusion templates were generated by fusion PCR which allows the joining of two PCR products from different chromosomes. For example, while generating a ALK intron 19 and EML4 intron 13 fusion template, EML4 intron 13 fragment was amplified with SEQ ID NO: 26 (5′-CTTCCTTCAGAGTAGG AGGTTC-3′) and SEQ ID NO: 27 (5′-ATTACATAGGGTGGGAGCCAAACCAGTATGAAACTCTGTGCAG TCATAAG-3′) which fused ALK sequence at the 5′ end. ALK intron 19 fragment was amplified with SEQ ID NO: 29 (5′-GATTCAGTGGGTAGATTCTGTGTG-3′) and SEQ ID NO: 28 (5′-CTTATGACTGCA CAGAGTTTCATACTG GTTTGGCTCCCACCCTATGTAAT-3′) which fused EML4 sequence at the 5′ end and is complimentary to SEQ ID NO: 27. Thermocycling of these two PCR amplifications with HiFi platinum Taq polymerase (Invitrogen) was performed as follows: denaturing at 94° C. for 2 min; PCR amplification was performed by 38 cycles of denaturation at 94° C. for 30 sec; annealing at 50-65° C. for 30 sec; extension at 68° C. for 1 min; and final extension at 68° C. for 5 min. Two PCR products were run on agarose gel to confirm the correct amplification, and purified by Qiagen® PCR cleanup columns, and the concentration was measured. The PCR products were diluted into about 1 ng/ul and mixed in a 1:1 ratio. A final PCR was set up by using the mixed diluted PCR product as template with primers of SEQ ID NO: 26 and 27, and thermocycling of the final PCR amplification with HiFi platinum Taq polymerase (Invitrogen) was performed as follows: denaturing at 94° C. for 2 min; PCR amplification was performed by 38 cycles of denaturation at 94° C. for 30 sec; annealing at 50-65° C. for 30 sec; extension at 68° C. for 2 min; and final extension at 68° C. for 5 min. The PCR product was run on agarose gel to confirm the correct ALK intron 19: EML4 intron 13 fusion amplification, and purified by Qiagen® PCR cleanup columns and the concentration was measured. Fusion templates mimicking the known ALK, ROS1 and RET translocations were generated by fusion PCR using this same general procedure. The fusion PCR products were diluted to 0.001 pg/ul and mixed with 0.1 pg/ul normal genomic DNA to mimic 1% translocation alleles in 10,000 copies of wild type alleles.

Using the mimicked fusion PCR products as templates, a multiplex translocation detection PCR according to the methods of the invention was set up with five ALK primers and 52 primers from partner genes in one reaction following the same thermocycling program described above. The primers described in Example 1 were employed together with the following additional primers covering all known ALK translocation partners: EML4 exon 20 and intron 20 (SEQ ID NO: 42-43), EML4 exon 14 and intron 14 (SEQ ID NO: 44-46), EML4 exon 15 (SEQ ID NO: 47), EML4 exon 18 and intron 18 (SEQ ID NO: 48-50), EML4 exon 2 and intron 2 (SEQ ID NO: 51-62), EML4 exon 17 and intron 17 (SEQ ID NO: 63-76), KIF5B exon 24 and intron 24 (SEQ ID NO: 77-79), KIF5B exon 17 and intron 17 (SEQ ID NO: 80-81), KIF5B exon 15 and intron 15 (SEQ ID NO: 82-88), KLC1 exon 9 and intron 9 (SEQ ID NO: 89-91), TFG exon 3 and intron 3 (SEQ ID NO: 92-103), SEC31A exon 21 and intron 21 (SEQ ID NO: 104-106), TPR exon 15 and intron 15 (SEQ ID NO: 107-108), SQSTM1 exon 5 and intron 5 (SEQ ID NO: 109-117), DCTN1 exon 26 and intron 26 (SEQ ID NO: 118-119), STRN exon 3 and intron 3 (SEQ ID NO: 120-129), PPFIBP1 exon 8 and intron 8 (SEQ ID NO: 130-132), PPFIBP1 exon 12 and intron 12 (SEQ ID NO: 133-137), and HIP1 exon 21, exon 28, exon 30 and intron 30 (SEQ ID NO: 138-141).

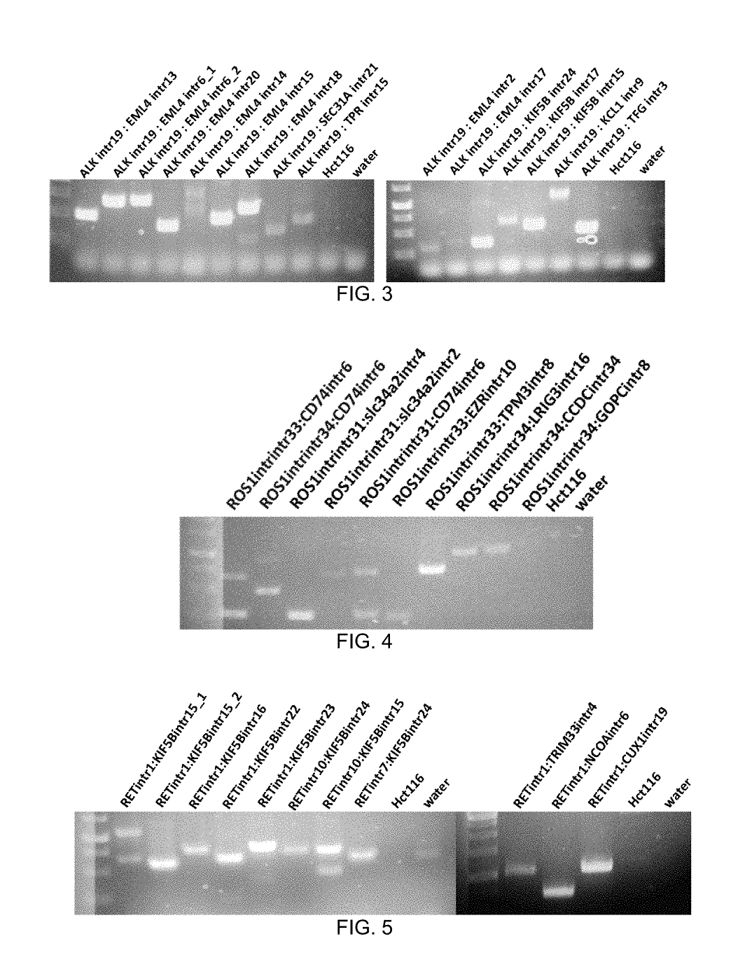

The fusion PCR products were used as templates to test the efficiency of the multiplex PCR method in detecting those translocations. All the indicated ALK translocation primers were included in two master mixes to amplify ALK translocations. The translocation-free human cancer cell line Hct116 was used as a negative control. All fusion PCR templates were spiked in Hct116 genomic DNA as 100%, 10%, 1%. Thermocycling was performed as follows: denaturing at 94° C. for 2 min; PCR amplification performed by 38 cycles of denaturation at 94° C. for 30 sec; annealing at 50-65° C. for 30 sec; extension at 68° C. for 2 min; and final extension at 68° C. for 5 min. 5% DMSO was included as an additive enhancer in multiplex PCR. One fifth of PCR products were run on 1% agarose gel and the obtained gel image is shown in FIG. 3.

FIG. 3 shows the results of two multiplex PCRs for detection of translocations between ALK and EML4, KIF5B, KCL1 and TFG. In the first multiplex PCR, five ALK primers and 52 primers from partner genes were included in the PCR. In second multiplex PCR, five ALK primers and 53 primers from partner genes were included in the PCR. About 100 copies of the respective translocation template in 10,000 copies of wild-type DNA (about 1%) were successfully detected by the multiplex PCR method of the invention.

In a further embodiment, the invention is directed to a kit for detecting the presence or absence of at least one ALK gene fusion in a tissue sample, the kit comprising reverse primers of SEQ ID NO: 1-5 and forward primers of SEQ ID NO: 6-141, as described above.

### Example 3

In this example, fusion products were generated and used as a sample in a multiplex PCR method according to the invention to detect ROS1 gene fusions. ROS1 intron 31, 33, 34 was fused, respectively, with all known partner introns using the general technique described in Example 2.

There are three ROS1 introns involved in ROS1 translocation in NSCLC. Reverse primers aligned on the known translocation introns were designed. Specifically, 15 reverse primers aligned on ROS1 exon 32 and intron 31 with a distance about 0.5-1 kb (SEQ ID NO: 142-156), four reverse primers on exon 34 and intron 33 (SEQ ID NO: 157-160), and five reverse primers on exon 35 and intron 34 (SEQ ID NO: 161-165) were employed. The following primers covering all known ROS1 translocation partners were employed: slc34a2 exon 4 and intron 4 (SEQ ID NO: 166-168), slc34a2 exon 12 and intron 12 (SEQ ID NO: 169-171), SDC4 exon 2 and intron 2 (SEQ ID NO: 172-176), SDC4 exon 4 and intron 4 (SEQ ID NO: 177-181), CD74 exon 6 and intron 6 (SEQ ID NO: 182-185), EZR exon 10 and intron 10 (SEQ ID NO: 186-187), LRIG3 exon 16 and intron 16 (SEQ ID NO: 188-190), TPM3 exon 2 and intron 2 (SEQ ID NO: 191-195), TPM3 exon 8 and intron 8 (SEQ ID NO: 196-197), GOPC exon 4 and intron 4 (SEQ ID NO: 198-200), GOPC exon 8 and intron 8 (SEQ ID NO: 201-205), and CCDC6 exon 6 and intron 6 (SEQ ID NO: 206-208).

The fusion PCR products were added as template to test the efficiency of the inventive multiplex PCR method in detection of the ROS1 translocations. All these ROS1 translocation primers were included in one master mix to amplify ROS1 translocations. The translocation-free human cancer cell line Hct116 was included as a negative control. All fusion PCR templates were spiked in Hct116 genomic DNA as 100%, 10%, 1%. Thermocycling was performed as follows: denaturing at 94° C. for 2 min; PCR amplification performed by 38 cycles of denaturation at 94° C. for 30 sec; annealing at 50-65° C. for 30 sec; extension at 68° C. for 2 min; and final extension at 68° C. for 5 min. 5% DMSO was included as an additive enhancer in multiplex PCR. PCR products were run on 1% agarose gel as shown in FIG. 4.

FIG. 4 shows the results of the single multiplex PCR detection of translocations between ROS1 and CD74, slc34a2, EZR, TPM3, LRIG3, CCDC and GOPC, respectively. The multiplex PCR employed twenty ROS1 primers and 37 primers from partner genes. About 100 copies of a respective translocation template in 10,000 copies of wild-type DNA (about 1%) were successfully detected by the multiplex PCR.

In a further embodiment, the invention is directed to a kit for detecting the presence or absence of at least one ROS1 gene fusion in a tissue sample, the kit comprising reverse primers of SEQ ID NO: 142-165 and forward primers of SEQ ID NO: 166-208, as described above.

### Example 4

In this example, fusion products were generated and used as a sample in a multiplex PCR method according to the invention to detect RET gene fusions. The fusion products were generated using the general technique described in Example 2.

There are three RET introns involved in RET translocation in NSCLC. In this example, four reverse primers aligned on RET exon 12 and intron 11 (SEQ ID 209-212) with a distance about 0.5-1 kb therebetween, two reverse primers on exon 11 and intron 10 (SEQ ID 213-214) with a distance about 0.5-1 kb therebetween, and two reverse primers on exon 8 and intron 7 (SEQ ID 215-216) with a distance about 0.5-1 kb therebetween, were employed. The following primers covering all known RET translocation partners were employed: KIF5B exon 15 and intron 15 (SEQ ID 217-226), KIF5B exon 16 and intron 16 (SEQ ID 227-228), KIF5B exon 22, exon 23 and intron 23 (SEQ ID 229-231), KIF5B exon 24 and intron 24 (SEQ ID 232-235), TRIM33 exon 14 and intron 14 (SEQ ID 236-237), NCOA4 exon 6 and intron 6 (SEQ ID 238-240), and CUX1 exon 19 and intron 19 (SEQ ID 241-251).

Fusion PCR products were generated by fusing RET intron 11, 10, 7 with all partner introns. The fusion PCR products were added as a template to test the efficiency of the multiplex PCR detection of those translocations according to the invention. The indicated RET translocation primers were included in two master mixes to amplify RET translocations. The translocation-free human cancer cell line Hct116 was added as a negative control. All fusion PCR templates were spiked in Hct116 genomic DNA as 100%, 10%, 1%. Thermocycling was performed as follows: denaturing at 94° C. for 2 min; PCR amplification performed by 38 cycles of denaturation at 94° C. for 30 sec; annealing at 50-65° C. for 30 sec; extension at 68° C. for 2 min; and final extension at 68° C. for 5 min. 5% DMSO was included as an additive enhancer in the multiplex PCR. The PCR products were run on 1% agarose gel as shown in FIG. 5.

FIG. 5 shows the results of the two multiplex PCR detection translocations between RET and KIF5B, TRIM33, NCOA and CUX1, respectively. In the first multiplex PCR, eight RET primers and 19 primers from partner genes were included in the PCR. In the second multiplex PCR, eight RET primers and 16 primers from partner genes were included in the PCR. About 100 copies of each translocation template in 10,000 copies wild-type DNA (about 1%) were successfully detected by the multiplex PCR according to the invention.

In a further embodiment, the invention is directed to a kit for detecting the presence or absence of at least one RET gene fusion in a tissue sample, the kit comprising reverse primers of SEQ ID NO: 209-216 and forward primers of SEQ ID NO: 217-251, as described above.

Examples 2-4 demonstrate that five multiplex PCRs may be conducted (two for ALK gene fusions, one for ROS1 gene fusions, and 2 for RET gene fusions) to detect all known ALK, ROS1 and RET translocations in NSCLC, thereby providing an efficient means for determining if a patient is appropriate for target therapy employing one or more drugs showing improved outcomes for treatment of NSCLC in which one or more of such gene fusions appear.

In some embodiments, translocations were detected by multiplex real time PCR run on the ABI StepOne plus instrument. In the real time PCR mixture, EvaGreen® was included without ROX reference dye (ROX was deselected as a reference dye during program setup on the ABI StepOne plus instrument). In light of the many primers included in PCR reaction, primer dimers more readily form, which show amplification in negative and blank controls. The multiplex PCR positive was determined by comparing a sample melt curve to a negative sample and a no template blank (not shown herein).

### Example 5

In some embodiments of the methods as described, translocation detection PCR in 50 ul was run on a PCR instrument using the following procedures. 10 ul of PCR products were mixed with I ul 20-fold diluted EvaGreen® and run on the ABI StepOne plus instrument without ROX reference dye to obtain a melt curve. The multiplex PCR positive was determined by comparing sample melt curve to negative sample and no template blank. 40 ul of the corresponding positive PCR products were gel purified with Qiagen® Gel Purification columns and sequenced with determined multiplex PCR primers from both ends. The exact translocation breakpoints in samples were obtained. Based on the translocation breakpoint sequence, two translocation PCR primers giving less than 100 bp product and spanning the breakpoint were designed. Also, the translocation PCR was amplified and purified, and product concentration was measured to make serial dilutions as standards in real time PCR.

10 ml of blood from the patient with the defined translocation was drawn. Cell-free plasma DNA was extracted using the Qiagen® QIAseq® cfDNA Extraction Kit and eluted in 30 ul of RNase and DNase-free water. A real-time PCR with SYBR® master mixture and the designed specific translocation primers on ABI StepOne plus real time PCR instrument was set up by using 10 ul of patient cfDNA and diluted translocation standard as templates to measure the copy number of the translocation allele in the sample. Meanwhile, an internal ERV3 loading real time PCR with SYBR® master mixture and the 10 ul of patient cfDNA was run on ABI StepOne plus real time PCR instrument. Patient plasma cfDNA translocation monitoring real time PCR could be performed, for example, every three weeks during targeting therapy.

### Example 6

In this example, two templates of ALK crizotinib-resistant mutations L1196M and G1269A were generated by fusion PCR. SEQ ID NO 252 (5′-GAAAGTTCTCCTCTGTGTTT GTCTCTAGTTTGG-3′) and SEQ ID NO 253 (5′-CCCTGCCCCGGTTCATCCTGATGGAGCT CATGGCGGGGGGA-3′) containing L1196M point mutation were used to amplify a 744 bp fragment upstream to point mutation site; SEQ ID NO 254 (5′-TCCCCCCGCCATGAGCTCCAtC AGGATGAACCGGGGCAGGG-3′) containing L1196M point mutation and complimentary to SEQ ID NO 253 and SEQ ID NO 255 (5′-GGCCCTACTGCCCTGTGTGTC-3′) were used to amplify a 631 bp fragment downstream to point mutation site. Two PCR products were purified, diluted and mixed in a 1:1 ratio as a template to set up fusion PCR using primers SEQ ID NO 252 and 255 to obtain a ALK L1196M template. Similarly, SEQ ID NO 256 (5′-CAACTGGCAGAAACCAGCCCGT-3′) and SEQ ID NO 257 (5′-TCTCGGGCCATCCCGAAGTCTGCAATCTTGGCCACTCTTCCAGGG-3′) containing G1269A point mutation were used to amplify a 539 bp fragment upstream to point mutation site; SEQ ID NO 258 (5′-CCCTGGAAGAGTGGCCAAGATTGcAGACTTCGGGATGGCCCGAGA-3′) containing G1269A point mutation and complimentary to SEQ ID NO 257 and SEQ ID NO 259 (5′-GCCACTTAGAATTCCTGAGTACTGAGG-3′) were used to amplify a 713 bp fragment downstream to point mutation site. Two PCR products were purified, diluted and mixed 1:1 as template to set up fusion PCR using primers SEQ ID NO 256 and 259 to obtain ALK G1269A template.

In the mutation-specific PCR, primers and probes designed for ALK L1196M mutation real-time PCR were mutation-specific primer SEQ ID NO 260 (5′-CC GCCATGAGCTCCAt-3′), SEQ ID NO 261 (5′-CCACCAGAACATTGTTCGCTGC-3′) and modified blocker probe SEQ ID NO 262 (5′-GCTCCAGCAGGATGAACC/3Phos/). Primers and probes designed for ALK G1269A mutation real-time PCR were mutation-specific primer SEQ ID NO 263 (5′-GAAGAGTGGCC AAGATTGc-3′), SEQ ID NO 264 (5′-CGGAGGGGTGAGGCAG-3′) and modified blocker probe SEQ ID NO 265 (5′-GCCAAGATTGGAGACTTCGG/3Phos/). In 20 ul of real time PCR reaction, 1 ul of serials diluted ALK L1196M or G1269A from 100,000 to 10 copies, 10 ul of 2×SYBR® green master mixture, 1 ul of 2 uM SEQ 260, 1 ul of 2 uM SEQ 261, 1 ul of 8 uM SEQ 262, 1 ul of 2 uM SEQ 263, 1 ul of 2 uM SEQ 264, 1 ul of 8 uM SEQ 265 and 3 ul of water were included. Multiplex mutation real time PCR was performed on ABI StepOne Plus instrument with a thermocycling program at 95° C. for 10 min; 95° C. for 15 sec, 50-65° C. for 30 sec, for 40 cycles; following melting curve 95° C. for 15 sec, 60° C. for 1 min, 95° C. for 15 sec with collection of the fluorescence signal per 0.3° C. ramping. The Ct values and melting temperature of two ALK mutations L1196M and G1269A are described in FIG. 7. The negative and no template controls showed amplification according to Ct measurement, but melting curves showed these are unspecific PCR primer dimers. The multiplex mutation-specific real time PCR results demonstrated as low as 0.01% mutated alleles (about one mutated copy in 10,000 wild type copies) could be detected by the invention methods.

Significantly, all ten known ALK crizotinib-resistant mutations can be detected in patient plasma cfDNA using two multiplex mutation specific real time PCRs according to the present methods as described above.

Significantly, all five known ROS1 crizotinib-resistant mutations can be detected in patient plasma cfDNA using two multiplex mutation specific real time PCRs according to the present methods as described above.

The examples and specific embodiments described herein are exemplary only in nature and are not intended to be limiting of the invention defined by the claims. Further embodiments and examples, and advantages thereof, will be apparent to one of ordinary skill in the art in view of this specification and are within the scope of the claimed invention.

What is claimed is:

1. A method for detecting the presence or absence of at least two known gene fusions in a tissue sample, wherein each gene fusion is formed between a respective first gene at a first fusion breakpoint location and a respective second gene at a second fusion breakpoint location, the method comprising
(a) providing genomic DNA isolated from the tissue sample,
(b) subjecting the isolated genomic DNA to multiplex PCR, wherein, for each known gene fusion, the multiplex PCR employs one or a plurality of forward primers which hybridize to the respective first gene adjacent the first fusion breakpoint location, wherein the plurality of forward primers hybridize to the respective first gene at consecutive respective positions along the first gene and are separated from one another by a first plurality of base pairs, and one or a plurality of reverse primers which hybridize to the respective second gene adjacent the second fusion breakpoint location, wherein the plurality of reverse primers hybridize to the respective second gene at consecutive respective positions along the second gene and are separated from one another by a second plurality of base pairs, and
(c) detecting if one or more amplified products are formed, each amplified product respectively representing the presence of a gene fusion.

2. The method of claim 1, wherein the first plurality of base pairs and the second plurality of base pairs are each from about 0.1 to about 4 kb, from about 0.25 to about 3 kb, or from about 0.5 to about 2 kb.

3. The method of claim 1, wherein the first plurality of base pairs and the second plurality of base pairs are each about 0.5 to about 1 kb.

4. The method of claim 1, for detecting the presence or absence of three or more known gene fusions in a tissue sample.

5. The method of claim 1, wherein the first gene of the respective gene fusions are selected from the group consisting of EML4, KIF5B, KLC1, TGF, SEC31A, TPR, SQSTM1, DCTN1, STRN, PPFIBP1 and HIP1 and the second gene of the respective gene fusions are ALK.

6. The method of claim 5, wherein the first fusion breakpoint location of the respective gene fusions are selected from the group consisting of EML4 intron 2, EML4 intron 6, EML4 intron 13, EML4 intron 14, EML4 intron 15, EML4 intron 18, EML4 intron 17, EML4 intron 20, KIF5B intron 24, KIF5B intron 17, KIF5B intron 15, KLC1 intron 9, TFG intron 3, SEC31A intron 21, TPR intron 15, SQSTM1 intron 5, DCTN1 intron 26, STRN intron 3, PPFIBP1 intron 8, PPFIBP1 intron 12, HIP1 intron 21, HIP1 intron 28, and HIP1 intron 30, and the second fusion breakpoint location of the respective gene fusions are ALK intron 19.

7. The method of claim 1, wherein the first gene of the respective gene fusions are selected from the group consisting of slc34a2, SDC4, CD74, EZR, LRIG3, TPM3, GOPC, and CCDC6, and the second gene of the respective gene fusions are ROS1.

8. The method of claim 7, wherein the first fusion breakpoint location of the respective gene fusions are selected from the group consisting of slc34a2 intron 4, slc34a2 intron 12, SDC4 intron 2, SDC4 intron 4, CD74 intron 6, EZR intron 10, LRIG3 intron 16, TPM3 intron 2, TPM3 intron 8, GOPC intron 4, GOPC intron 8, and CCDC6 intron 6, and the second fusion breakpoint location of the respective gene fusions are selected from the group consisting of ROS1 intron 31, ROS1 intron 33, and ROS1 intron 34.

9. The method of claim 1, wherein the first gene of the respective gene fusions are selected from the group consisting of KIF5B, TRIM33, NCOA4 and CUX1, and the second gene of the respective gene fusions are RET.

10. The method of claim 9, wherein the first fusion breakpoint location of the respective gene fusions are selected from the group consisting of KIF5B intron 15, KIF5B intron 16, KIF5B intron 23, KIF5B intron 24, TRIM33 intron 14, NCOA4 intron 6, and CUX1 intron 19, and the second fusion breakpoint location of the respective gene fusions are selected from the group consisting of RET intron 11, RET intron 10, and RET intron 7.

11. The method of claim 1, for detecting the presence or absence of at least three or more gene fusions, wherein two or more multiplex PCR according to step (c) are performed and wherein the primers for the respective known gene fusions are divided among the multiplex PCRs to minimize dimer formation between primers.

12. The method of claim 1, wherein at least one amplified product is detected in step (c), the method further comprising
(d) subjecting genomic DNA isolated from the tissue sample to an individual PCR for each gene fusion for which forward primer(s) and reverse primer(s) were employed in step (b), the respective individual PCRs employing the forward primer(s) and the reverse primer(s) employed in the multiplex PCR for the respective gene fusion; and
(e) in each individual PCR, detecting if an amplified product is formed, the amplified product representing the presence of the respective gene fusion.

13. The method of claim 12, further comprising sequencing an amplified product detected in step (e), and designing a forward primer and a reverse primer around the gene fusion in the amplified product, operable to amplify a fragment encompassing the gene fusion.

14. The method of claim 13, wherein the fragment comprises from about 50 to about 150 base pairs.

15. The method of claim 13, wherein sequencing an amplified product detected in step (e) is conducted by Sanger sequencing.

16. A method for assessing cancer progression or cancer regression during targeted treatment, comprising determining the presence of a gene fusion in a patient's tissue sample and designing a forward primer and a reverse primer around the gene fusion operable to amplify a fragment encompassing the gene fusion, according to claim 13, and monitoring the amount of fusion product in patient plasma cell-free DNA (cfDNA) using the designed primers in a PCR during the targeted treatment.

17. A method for monitoring a patient undergoing targeted therapy with a drug to which resistance may develop, comprising detecting the presence or absence of one or more acquired functional mutations associated with the drug resistance in a tissue sample from the patient according to the method of claim 1.

18. The method of claim 17, wherein the step of detecting the presence or absence of one or more acquired functional mutations associated with the drug resistance is repeated at least once during the targeted therapy.

19. The method of claim 17, wherein the drug is crizotinib and the step of detecting the presence or absence of one or more acquired functional mutations associated with the drug resistance detects the presence or absence of crizotinib-resistant ALK mutations and crizotinib-resistant ROS1 mutations.

20. The method of claim 16, wherein the genomic DNA is plasma cell-free DNA.

21. The method of claim 17, wherein
(a) the crizotinib-resistant ALK mutations are selected from 1511Tins, L1152R, C1156Y, 11171T, 11171S, F1174V, F1174C, L1196M, L1198F, G1202R, S1206Y and G1269A, and combinations thereof; or
(b) the crizotinib-resistant ROS1 mutations are selected from L2026M, G2032R, D2032N, L2155S, S1986Y and S1986F, and combinations thereof.

22. The method of claim 1, wherein at least one of the known gene fusions is BCR-ABL1, RBN15-MKL1, NPM1-ALK, IGH-MYC, RUNX1-RUNX1T1, ETV6-RUNX1, IGH-MAF, PML-RARA, FGFR2-KIAA1967, FGFR3-TACC3, CD74-NTRK1, MPRIP-NTRK1, TPM3-NTRK1, LMNA-NTRK1, SQSTM1-NTRK1, TPR-NTRK1, PEAR1-NTRK1, IRF2BP2-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, QKI-NTRK2, NACC2-NTRK2, VCL-NTRK2, AGBL4-NTRK2, PAN3-NTRK2, AFAP1-NTRK2, DAB21P-NTRK2, TRIM24-NTRK2, SQSTM1-NTRK2, ETV6-NTRK3, BTBD1-NTRK3, EML4-NTRK3, TFG-NTRK3, RBPMS-NTRK3, or LYN-NTRK3.

23. A method for detecting the presence or absence of a known gene fusion in a tissue sample, wherein the gene fusion is formed between a first gene at a first fusion breakpoint location and a second gene at a second fusion breakpoint location, the method comprising
(a) providing genomic DNA isolated from the tissue sample,
(b) subjecting the isolated genomic DNA to PCR amplification, the PCR amplification employing one or a plurality of forward primers which hybridize to the respective first gene adjacent the first fusion breakpoint location, wherein the plurality of forward primers hybridize to the respective first gene at consecutive respective positions along the first gene and are separated from one another by a first plurality of base pairs, and one or a plurality of reverse primers which hybridize to the respective second gene adjacent the second fusion breakpoint location, wherein the plurality of reverse primers hybridize to the respective second gene at consecutive respective positions along the second gene and are separated from one another by a second plurality of base pairs, and
(c) detecting if an amplified product is formed, the amplified product representing the presence of the gene fusion.

24. The method of claim 23, wherein the first plurality of base pairs and the second plurality of base pairs are each from about 0.1 to about 4 kb, from about 0.25 to about 3 kb, or from about 0.5 to about 2 kb.

25. The method of claim 24, wherein the first plurality of base pairs and the second plurality of base pairs are each from about 0.5 to about 1 kb.

26. The method of claim 23, wherein
(a) the first gene is EML4, KIF5B, TGF, SEC31A, TPR, SQSTM1, DCTN1, STRM, or PPFIBP1, and the second gene is ALK,
(b) the first gene is slc34a2, SDC4, CD74, EZR, LRIG3, TPM3, GOPC OR CCDC6, and the second gene is ROS1;
(c) the first gene is KIF5B, TRIM33, NCOA4 or CUX1, and the second gene is RET; or
(d) the gene fusion is BCR-ABL1, RBN15-MKL1, NPM1-ALK, IGH-MYC, RUNX1-RUNX1T1, ETV6-RUNX1, IGH-MAF, PML-RARA, FGFR2-KIAA1967, FGFR3-TACC3, CD74-NTRK1, MPRIP-NTRK1, TPM3-NTRK1, LMNA-NTRK1, SQSTM1-NTRK1, TPR-NTRK1, PEAR1-NTRK1, IRF2BP2-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, QKI-NTRK2, NACC2-NTRK2, VCL-NTRK2, AGBL4-NTRK2, PAN3-NTRK2, AFAP1-NTRK2, DAB21P-NTRK2, TRIM24-NTRK2, SQSTM1-NTRK2, ETV6-NTRK3, BTBD1-NTRK3, EML4-NTRK3, TFG-NTRK3, RBPMS-NTRK3, or LYN-NTRK3.

27. The method of claim 23, further comprising sequencing the amplified product detected in step (c) by Sanger sequencing, and designing a forward primer and a reverse primer around the gene fusion operable to amplify a fragment encompassing the gene fusion.

28. The method of claim 27, wherein the fragment comprises from about 50 to about 150 base pairs.

29. A method for assessing cancer progression or cancer regression during targeted treatment, comprising determining the presence of a gene fusion in a patient's tissue sample and designing a forward primer and a reverse primer around the gene fusion operable to amplify a fragment encompassing the gene fusion, according to claim 27, and monitoring the amount of fusion product in patient plasma cell-free DNA (cfDNA) using the designed primers in a PCR during the targeted treatment.

30. A method for monitoring a patient undergoing targeted therapy with a drug to which resistance may develop, comprising detecting the presence or absence of one or more acquired functional mutations associated with the drug resistance in a tissue sample from the patient according to the method of claim 23.

31. The method of claim 30, wherein the step of detecting the presence or absence of the one or more acquired functional mutations associated with the drug resistance is repeated at least once during the targeted therapy.

32. The method of claim 30, wherein the drug is crizotinib and the step of detecting the presence or absence of the one or more acquired functional mutations associated with the drug resistance detects the presence or absence of a crizotinib-resistant ALK mutation or a crizotinib-resistant ROS1 mutation.

33. The method of claim 29, wherein the genomic DNA is plasma cell-free DNA.

34. A kit for detecting the presence or absence of a gene fusion in a tissue sample, wherein
(a) the gene fusion is at least one ALK gene fusion, the kit comprising reverse primers of SEQ ID NO: 1-5 and forward primers of SEQ ID NO: 6-141;
(b) the gene fusion is at least one ROS1 gene fusion, the kit comprising reverse primers of SEQ ID NO: 142-165 and forward primers of SEQ ID NO:166-208; or
(c) the gene fusion is at least one RET gene fusion, the kit comprising reverse primers of SEQ ID NO: 209-216 and forward primers of SEQ ID NO: 217-251.

35. A kit according to claim 34, further comprising a negative control isolated DNA sample which is free of a positive fusion DNA.

36. A composition comprising an isolated DNA sample from a patient non-small cell lung cancer tumor, and
(a) reverse primers of SEQ ID NO: 1-5, and forward primers of SEQ ID NO: 6-141;
(b) reverse primers of SEQ ID NO: 142-165 and forward primers of SEQ ID NO:166-208; or
(c) reverse primers of SEQ ID NO: 209-216 and forward primers of SEQ ID NO: 217-251.

37. A composition according to claim 36, further comprising a polymerase.


SEQUENCE LISTING (265 sequences)

SEQ ID NO: 1 - DNA, 21 bp, Homo sapiens
gcagtagttggggttgtagtc

SEQ ID NO: 2 - DNA, 26 bp, Homo sapiens
gctctgaacctttccatcatacttag

SEQ ID NO: 3 - DNA, 21 bp, Homo sapiens
gagttggagaagagccacatc

SEQ ID NO: 4 - DNA, 20 bp, Homo sapiens
tgctgccatctcccttctac

SEQ ID NO: 5 - DNA, 20 bp, Homo sapiens
gttgggagcttccgttttgg

SEQ ID NO: 6 - DNA, 20 bp, Homo sapiens
tactgtagagcccacacctg

SEQ ID NO: 7 - DNA, 21 bp, Homo sapiens
caggtcctctttgcccttttc

SEQ ID NO: 8 - DNA, 20 bp, Homo sapiens
gcacagtgtctggcatttgc

SEQ ID NO: 9 - DNA, 21 bp, Homo sapiens
gaagaatggcagtgggttgag

SEQ ID NO: 10 - DNA, 20 bp, Homo sapiens
cctgtggcagtcttaccaac

SEQ ID NO: 11 - DNA, 20 bp, Homo sapiens
ggcaggcagtgtaaacttgc

SEQ ID NO: 12 - DNA, 23 bp, Homo sapiens
gagaactatgaaactcccacacc

SEQ ID NO: 13 - DNA, 20 bp, Homo sapiens
agcaagccctttcctgtctc

SEQ ID NO: 14 - DNA, 25 bp, Homo sapiens
caagaggactaagggctgtttattg

SEQ ID NO: 15 - DNA, 23 bp, Homo sapiens
cctcagaaggtcaaccaaagatg

SEQ ID NO: 16 - DNA, 27 bp, Homo sapiens
ggcataaagatgtcatcatcaaccaag

SEQ ID NO: 17 - DNA, 25 bp, Homo sapiens
gtgatttgagctagatgaatccagc

SEQ ID NO: 18 - DNA, 22 bp, Homo sapiens
ctcagtttcctgtcttgccaac

SEQ ID NO: 19 - DNA, 27 bp, Homo sapiens
ctttagtaggtcttactttctcccttc

SEQ ID NO: 20 - DNA, 29 bp, Homo sapiens
gaaatatagtggcatctactgaaataggg

SEQ ID NO: 21 - DNA, 23 bp, Homo sapiens
gtggcttgttccaaatcatctgc

SEQ ID NO: 22 - DNA, 20 bp, Homo sapiens
gggacttcaggcaccatgta

SEQ ID NO: 23 - DNA, 22 bp, Homo sapiens
ctggaggagaatctgaatgtcc

SEQ ID NO: 24 - DNA, 28 bp, Homo sapiens
cattctaacaggttacagttctgatgtg

SEQ ID NO: 25 - DNA, 26 bp, Homo sapiens
gttaggggtaaaatgacctgtatcac

SEQ ID NO: 26 - DNA, 21 bp, Homo sapiens
gcaggtgcttttgttcagagc

SEQ ID NO: 27 - DNA, 21 bp, Homo sapiens
gaaaggacggaagaccaatgc

SEQ ID NO: 28 - DNA, 23 bp, Homo sapiens
gttgtggttagtagcagtcttcc

SEQ ID NO: 29 - DNA, 20 bp, Homo sapiens
cgtccctgaactacactcac

SEQ ID NO: 30 - DNA, 20 bp, Homo sapiens
ttgagaaagcagagccaggg

SEQ ID NO: 31 - DNA, 29 bp, Homo sapiens
catgcttacagattccattgtttcattgg

SEQ ID NO: 32 - DNA, 22 bp, Homo sapiens
cgaagttctgctgttgcctttg

SEQ ID NO: 33 - DNA, 25 bp, Homo sapiens
gcagaaacatcttcctcttctactg

SEQ ID NO: 34 - DNA, 22 bp, Homo sapiens
gtctggggaaatgaacaacagc

SEQ ID NO: 35 - DNA, 22 bp, Homo sapiens
cccaaggtaaagagtgtcttcc

SEQ ID NO: 36 - DNA, 25 bp, Homo sapiens
gcactacatcatgcctttgcttttc

SEQ ID NO: 37 - DNA, 24 bp, Homo sapiens
gtgatgttgaaaccatagtgtggg

SEQ ID NO: 38 - DNA, 21 bp, Homo sapiens
ggttgtatcagcctctggttg

SEQ ID NO: 39 - DNA, 23 bp, Homo sapiens
ttgtatgtaggtcactgggatgc

SEQ ID NO: 40 - DNA, 26 bp, Homo sapiens
gatacctatgctgccaataaggaaac

SEQ ID NO: 41 - DNA, 20 bp, Homo sapiens
catccagtagcgtggtagac

SEQ ID NO: 42 - DNA, 21 bp, Homo sapiens
catcacacaccttgactggtc

SEQ ID NO: 43 - DNA, 23 bp, Homo sapiens
cagggaggaatatgatagatggg

SEQ ID NO: 44 - DNA, 20 bp, Homo sapiens
actggaggagggaaagacag

SEQ ID NO: 45 - DNA, 24 bp, Homo sapiens
gttcactccttcctttcccatttc

SEQ ID NO: 46 - DNA, 31 bp, Homo sapiens
atgtgatttcatgtgagctaatagaacttac

SEQ ID NO: 47 - DNA, 20 bp, Homo sapiens
gctgtagcagaaggaaaggc

SEQ ID NO: 48 - DNA, 21 bp, Homo sapiens
ctatccacacagacgggaatg

SEQ ID NO: 49 - DNA, 23 bp, Homo sapiens
ggcccttcaagtcctttagaatc

SEQ ID NO: 50 - DNA, 28 bp, Homo sapiens
gacgtggactttattgaccatttgttac

SEQ ID NO: 51 - DNA, 21 bp, Homo sapiens
gctcttgagtcacgagttcag

SEQ ID NO: 52 - DNA, 20 bp, Homo sapiens
gatagtgcagtcagccaacc

SEQ ID NO: 53 - DNA, 21 bp, Homo sapiens
caatgacaaaggacaaggggc

SEQ ID NO: 54 - DNA, 25 bp, Homo sapiens
gtctctaaagtgagtctaggatcag

SEQ ID NO: 55 - DNA, 21 bp, Homo sapiens
caggacacttgactgaggtag

SEQ ID NO: 56 - DNA, 20 bp, Homo sapiens
aggggatgatggagtgtctg

SEQ ID NO: 57 - DNA, 22 bp, Homo sapiens
gagaaaagtgacgaagagcctg

SEQ ID NO: 58 - DNA, 21 bp, Homo sapiens
ctcctctctgtgaactatccc

SEQ ID NO: 59 - DNA, 21 bp, Homo sapiens
gcatgtggtaggtcattctgg

SEQ ID NO: 60 - DNA, 26 bp, Homo sapiens
gaggtaacacttaataccctcttgag

SEQ ID NO: 61 - DNA, 22 bp, Homo sapiens
caaggtatggcttctttggagg

SEQ ID NO: 62 - DNA, 26 bp, Homo sapiens
cttatgatgttgtgtcttctgaccac

SEQ ID NO: 63 - DNA, 20 bp, Homo sapiens
ataggaacgcactcaggcag

SEQ ID NO: 64 - DNA, 24 bp, Homo sapiens
gaagctgatgtaacaccattgagc

SEQ ID NO: 65 - DNA, 20 bp, Homo sapiens
ccatctgaggtgtagtgctg

SEQ ID NO: 66 - DNA, 20 bp, Homo sapiens
ggcagtccatttggggattg

SEQ ID NO: 67 - DNA, 27 bp, Homo sapiens
gagtaagcacagtgtgaataaagcatc

SEQ ID NO: 68 - DNA, 33 bp, Homo sapiens
gttaatgctcacatagaaaggattctttttaac

SEQ ID NO: 69 - DNA, 24 bp, Homo sapiens
cattttaagaccaggcacaatggc

SEQ ID NO: 70 - DNA, 29 bp, Homo sapiens
gttgtaatcattatgggattctgtaaggc

SEQ ID NO: 71 - DNA, 20 bp, Homo sapiens
ctcagggagtgtccaaagag

SEQ ID NO: 72 - DNA, 20 bp, Homo sapiens
gcatgtggcttcaaagtgcc

SEQ ID NO: 73 - DNA, 21 bp, Homo sapiens
ctggcctgcctgtcagaaaac

SEQ ID NO: 74 - DNA, 24 bp, Homo sapiens
ccacgttaaaccataccactaagc

SEQ ID NO: 75 - DNA, 23 bp, Homo sapiens
ccatcccatccattttcttcctg

SEQ ID NO: 76 - DNA, 21 bp, Homo sapiens
gcaaagagacaggaatggcag

SEQ ID NO: 77 - DNA, 22 bp, Homo sapiens
cgcaaacgctatcagcaagaag

SEQ ID NO: 78 - DNA, 31 bp, Homo sapiens
ttggtgcaggaagtgttttttttgtgtttta

SEQ ID NO: 79 - DNA, 25 bp, Homo sapiens
ggtcctagagaaaaggatgttacag

SEQ ID NO: 80 - DNA, 20 bp, Homo sapiens
cgatgccctcagtgaagaac

SEQ ID NO: 81 - DNA, 22 bp, Homo sapiens
ggatttcccacagttacgaagc

SEQ ID NO: 82 - DNA, 23 bp, Homo sapiens
gcagaaataggaattgctgtggg

SEQ ID NO: 83 - DNA, 31 bp, Homo sapiens
atttataatgaatgggcttgtggttgcttta

SEQ ID NO: 84 - DNA, 25 bp, Homo sapiens
gttactgtttgttgatggaggcaag

SEQ ID NO: 85 - DNA, 35 bp, Homo sapiens
gttgttgttcttaagttacagtttacagttttatg

SEQ ID NO: 86 - DNA, 23 bp, Homo sapiens
attcatgtgtaggctgagcatgg

SEQ ID NO: 87 - DNA, 22 bp, Homo sapiens
ctggccctgttgttgagttttg

SEQ ID NO: 88 - DNA, 22 bp, Homo sapiens
cagtgaccacaattccttctgg

SEQ ID NO: 89 - DNA, 26 bp, Homo sapiens
caaggaaagttcaagcaagcagaaac

SEQ ID NO: 90 - DNA, 21 bp, Homo sapiens
ggtggctttttcctccatagc

SEQ ID NO: 91 - DNA, 29 bp, Homo sapiens
catgctgtaagaagtagtatctaacaagg

SEQ ID NO: 92 - DNA, 24 bp, Homo sapiens
ctatgggagagtccaactatgttc

SEQ ID NO: 93 - DNA, 31 bp, Homo sapiens
cttaagggacaagatagaaaagattacagag

SEQ ID NO: 94 - DNA, 25 bp, Homo sapiens
ggtggtgatagtagagattttagcc

SEQ ID NO: 95 - DNA, 32 bp, Homo sapiens
aaaacaaaacaaaacgagaacataagagcaag

SEQ ID NO: 96 - DNA, 20 bp, Homo sapiens
gcttggtcagtaggctatgc

SEQ ID NO: 97 - DNA, 25 bp, Homo sapiens
ctggcttctttagagactattggac

SEQ ID NO: 98 - DNA, 31 bp, Homo sapiens
gaataaaagggaattcttttttgtgaggcag

SEQ ID NO: 99 - DNA, 27 bp, Homo sapiens
ggcatttattacgtagaacaactgctg

SEQ ID NO: 100 - DNA, 31 bp, Homo sapiens
gatgggtgtttagtaagtttatcttgttgtg

SEQ ID NO: 101 - DNA, 25 bp, Homo sapiens
gttctggtaagatgtatcagtgtgc

SEQ ID NO: 102 - DNA, 25 bp, Homo sapiens
ggtgcttcaggagtttgtatgtatg

SEQ ID NO: 103 - DNA, 24 bp, Homo sapiens
caagagggataacagacctatcag

SEQ ID NO: 104 - DNA, 21 bp, Homo sapiens
taccaccttatccacagccac

SEQ ID NO: 105 - DNA, 32 bp, Homo sapiens
gaatgtgttctgtgctaaaatgaatgttattg

SEQ ID NO: 106 - DNA, 24 bp, Homo sapiens
cctaatctgggtttaggtctttgc

SEQ ID NO: 107 - DNA, 23 bp, Homo sapiens
caaacttgagagtgcccttactg

SEQ ID NO: 108 - DNA, 25 bp, Homo sapiens
caatagccactctaatgatggtagc

SEQ ID NO: 109 - DNA, 20 bp, Homo sapiens
cttctggtccatcggaggat

SEQ ID NO: 110 - DNA, 23 bp, Homo sapiens
cttctagttcaagatgacggtgg

SEQ ID NO: 111 - DNA, 20 bp, Homo sapiens
aacagtggaacaccgaggtc

SEQ ID NO: 112 - DNA, 24 bp, Homo sapiens
ggcttagactgttgtggaagttag

SEQ ID NO: 113 - DNA, 20 bp, Homo sapiens
tctgcttacaggcgagtctc

SEQ ID NO: 114 - DNA, 20 bp, Homo sapiens
tgctgagggaggttgaatgc

SEQ ID NO: 115 - DNA, 26 bp, Homo sapiens
gccattatcttctagattgaaagcgg

SEQ ID NO: 116 - DNA, 22 bp, Homo sapiens
gcacagaggtattaggaaacgg

SEQ ID NO: 117 - DNA, 20 bp, Homo sapiens
gcagatgtgaactccagcag

SEQ ID NO: 118 - DNA, 22 bp, Homo sapiens
gcagagaaggcagaactaaagc

SEQ ID NO: 119 - DNA, 24 bp, Homo sapiens
gacaaagtctctgttactgacacc

SEQ ID NO: 120 - DNA, 23 bp, Homo sapiens
gaagcctccaagctatgattctg

SEQ ID NO: 121 - DNA, 30 bp, Homo sapiens
acttattagtaggtattagggaagaatgac

SEQ ID NO: 122 - DNA, 22 bp, Homo sapiens
gagtgtctgttcttgtgacctg

SEQ ID NO: 123 - DNA, 27 bp, Homo sapiens
gtttttcagacctagtaccaaatccag

SEQ ID NO: 124 - DNA, 24 bp, Homo sapiens
ccaggagagaatgtaggaagttag

SEQ ID NO: 125 - DNA, 25 bp, Homo sapiens
cgtccagaccttcatatttctactg

SEQ ID NO: 126 - DNA, 21 bp, Homo sapiens
ggaaagaactgcgagcttagc

SEQ ID NO: 127 - DNA, 31 bp, Homo sapiens
catctgaagtcttgaaatggcaatgtaaatg

SEQ ID NO: 128 - DNA, 22 bp, Homo sapiens
ccagtccctattgtatctccac

SEQ ID NO: 129 - DNA, 22 bp, Homo sapiens
ggctcttctaccatcattcctc

SEQ ID NO: 130 - DNA, 23 bp, Homo sapiens
ggacagtgagagacttcagtatg

SEQ ID NO: 131 - DNA, 23 bp, Homo sapiens
gtttcgtatccacagtagaggtc

SEQ ID NO: 132 - DNA, 28 bp, Homo sapiens
gattctgtccttttgtcttagtgatgag

SEQ ID NO: 133 - DNA, 20 bp, Homo sapiens
cttcgacagtgcctgaacag

SEQ ID NO: 134 - DNA, 21 bp, Homo sapiens
gagtgcctctctaaggagttg

SEQ ID NO: 135 - DNA, 20 bp, Homo sapiens
ctcgcaacgtaagcactgga

SEQ ID NO: 136 - DNA, 30 bp, Homo sapiens
gcaaatgaacctctaataatcagtgttgac

SEQ ID NO: 137 - DNA, 24 bp, Homo sapiens
caagagcacagcagtgatgaaaac

SEQ ID NO: 138 - DNA, 23 bp, Homo sapiens
gacttctccattccataaccctg

SEQ ID NO: 139 - DNA, 23 bp, Homo sapiens
gattcagtccttgttgctctagg

SEQ ID NO: 140 - DNA, 22 bp, Homo sapiens
gcagaaggagcgtcaaaaactg

SEQ ID NO: 141 - DNA, 21 bp, Homo sapiens
caggtgctgagaaatggagtg

SEQ ID NO: 142 - DNA, 29 bp, Homo sapiens
gctcatctcagagttaaggtatatctttc

SEQ ID NO: 143 - DNA, 25 bp, Homo sapiens
gttttggagcattttccctccattc

SEQ ID NO: 144 - DNA, 20 bp, Homo sapiens
gttgagggataggagctgag

SEQ ID NO: 145 - DNA, 20 bp, Homo sapiens
actgggcccattccttaacc

SEQ ID NO: 146 - DNA, 20 bp, Homo sapiens
taccatctcacgcgagtcag

SEQ ID NO: 147 - DNA, 22 bp, Homo sapiens
ccccatggacaggaataatgag

SEQ ID NO: 148 - DNA, 24 bp, Homo sapiens
gagacacaacaacaaactttgggc

SEQ ID NO: 149 - DNA, 24 bp, Homo sapiens
ccagaaagatctcaaatcgtcacc

SEQ ID NO: 150 - DNA, 22 bp, Homo sapiens
gacttagactgccacgcaatag

SEQ ID NO: 151 - DNA, 21 bp, Homo sapiens
ccctgcaacagctcctgaaat

SEQ ID NO: 152 - DNA, 21 bp, Homo sapiens
gaaacacgtgggaaaacaccc

SEQ ID NO: 153 - DNA, 21 bp, Homo sapiens
aaggcctaaagtgaaccccag

SEQ ID NO: 154 - DNA, 21 bp, Homo sapiens
ggggagaaatgcagcatccat

SEQ ID NO: 155 - DNA, 20 bp, Homo sapiens
aaggaagagcagtgagccca

SEQ ID NO: 156 - DNA, 24 bp, Homo sapiens
cttcagctttctcccactgtattg

SEQ ID NO: 157 - DNA, 23 bp, Homo sapiens
ccaaaggtcagtgggattgtaac

SEQ ID NO: 158 - DNA, 26 bp, Homo sapiens
cttaggtagtttcagttgtgtagagg

SEQ ID NO: 159 - DNA, 21 bp, Homo sapiens
gcagtatgcgtaagtcaaggg

SEQ ID NO: 160 - DNA, 22 bp, Homo sapiens
gggggtggctgattattattgg

SEQ ID NO: 161 - DNA, 20 bp, Homo sapiens
gcatagcaggcattagccag

SEQ ID NO: 162 - DNA, 29 bp, Homo sapiens
gcagagtacaagagtgtttatcattgttg

SEQ ID NO: 163 - DNA, 22 bp, Homo sapiens
gctatttgtcttcccacacagg

SEQ ID NO: 164 - DNA, 28 bp, Homo sapiens
gcttcaaagtgctctcataaagattgtg

SEQ ID NO: 165 - DNA, 23 bp, Homo sapiens
atcaaagattgtcactggcctcc

SEQ ID NO: 166 - DNA, 21 bp, Homo sapiens
gagagagacaccaaagggaag

SEQ ID NO: 167 - DNA, 20 bp, Homo sapiens
cattctttcccagagccctc

SEQ ID NO: 168 - DNA, 20 bp, Homo sapiens
ggctaagggtggatagagag

SEQ ID NO: 169 - DNA, 20 bp, Homo sapiens
aggtagaagacggcgaacca

SEQ ID NO: 170 - DNA, 21 bp, Homo sapiens
gaccagcactgtccaatacac

SEQ ID NO: 171 - DNA, 26 bp, Homo sapiens
cttgcacaaagtaacatagatggtcc

SEQ ID NO: 172 - DNA, 23 bp, Homo sapiens
taccagacgatgaggatgtagtg

SEQ ID NO: 173 - DNA, 20 bp, Homo sapiens
ttagaagtggggatggggtg

SEQ ID NO: 174 - DNA, 21 bp, Homo sapiens
cacatagtcgcccaggaaatg

SEQ ID NO: 175 - DNA, 27 bp, Homo sapiens
gttacgagcccattattgggaaaaatg

SEQ ID NO: 176 - DNA, 23 bp, Homo sapiens
tttagagctgatgaagcctctgc

SEQ ID NO: 177 - DNA, 21 bp, Homo sapiens
gaggagaatgaggttatcccc

SEQ ID NO: 178 - DNA, 23 bp, Homo sapiens
gagttctttactggaatgtgcgg

SEQ ID NO: 179 - DNA, 20 bp, Homo sapiens
taatcagcacccaggagcct

SEQ ID NO: 180 - DNA, 20 bp, Homo sapiens
tcctctcggagcctgaaagt

SEQ ID NO: 181 - DNA, 24 bp, Homo sapiens
catacctgtctatgaagtgtaggc

SEQ ID NO: 182 - DNA, 22 bp, Homo sapiens
gtttgaaatgagcaggcactcc

SEQ ID NO: 183 - DNA, 21 bp, Homo sapiens
agtagaaggtcaaagggccac

SEQ ID NO: 184 - DNA, 20 bp, Homo sapiens
aagccacttgggaagaaggc

SEQ ID NO: 185 - DNA, 21 bp, Homo sapiens
agcccccattacacctctttg

SEQ ID NO: 186 - DNA, 20 bp, Homo sapiens
gagaaaccgtggagagagag

SEQ ID NO: 187 - DNA, 23 bp, Homo sapiens
gactaaaacccaaaggcgtgttc

SEQ ID NO: 188 - DNA, 21 bp, Homo sapiens
aagccaccaccagtttgtcac

SEQ ID NO: 189 - DNA, 21 bp, Homo sapiens
gacccttgctttcaatgctgc

SEQ ID NO: 190 - DNA, 20 bp, Homo sapiens
tcttgctgcccagtttctgc

SEQ ID NO: 191 - DNA, 20 bp, Homo sapiens
tgaagaagagctggaccgtg

SEQ ID NO: 192 - DNA, 21 bp, Homo sapiens
gaatcataggtaggggtgagg

SEQ ID NO: 193 - DNA, 23 bp, Homo sapiens
ctaagggtacgttactactgtgc

SEQ ID NO: 194 - DNA, 25 bp, Homo sapiens
gagtgtggaaaacaagttgcagaac

SEQ ID NO: 195 - DNA, 28 bp, Homo sapiens
cttggccaggatgtattacctttataac

SEQ ID NO: 196 - DNA, 20 bp, Homo sapiens
cgtgctgagtttgctgagag

SEQ ID NO: 197 - DNA, 20 bp, Homo sapiens
gttggtaactgccatccacc

SEQ ID NO: 198 - DNA, 26 bp, Homo sapiens
gaaagaagcacaacttgaagctgaag

SEQ ID NO: 199 - DNA, 20 bp, Homo sapiens
tgccttgagaccagtacagc

SEQ ID NO: 200 - DNA, 25 bp, Homo sapiens
ccttgaggatacctgttcttttcag

SEQ ID NO: 201 - DNA, 24 bp, Homo sapiens
cgtagagtatgaagatgagagtgg

SEQ ID NO: 202 - DNA, 30 bp, Homo sapiens
ttggtttagttcctttgctatcatttagac

SEQ ID NO: 203 - DNA, 26 bp, Homo sapiens
ggaattgctacataacttcatggtcc

SEQ ID NO: 204 - DNA, 21 bp, Homo sapiens
ctgtcgggattagaaagcctg

SEQ ID NO: 205 - DNA, 22 bp, Homo sapiens
gatacctgccataagagggaag

SEQ ID NO: 206 - DNA, 21 bp, Homo sapiens
cacagtatctggaggaggaac

SEQ ID NO: 207 - DNA, 25 bp, Homo sapiens
cgggataaggtagtggtaagataac

SEQ ID NO: 208 - DNA, 26 bp, Homo sapiens
ccttttgtaggtcagattatccacag

SEQ ID NO: 209 - DNA, 21 bp, Homo sapiens
ccgttgccttgaccacttttc

SEQ ID NO: 210 - DNA, 24 bp, Homo sapiens
caaagttctatccacacattgggc

SEQ ID NO: 211 - DNA, 20 bp, Homo sapiens
tccttggctcacacccttac

SEQ ID NO: 212 - DNA, 21 bp, Homo sapiens
atggaaatgggggcagaacac

SEQ ID NO: 213 - DNA, 20 bp, Homo sapiens
tgaggagatgggtggcttgt

SEQ ID NO: 214 - DNA, 20 bp, Homo sapiens
ctggcaggggaggtcaaaat

SEQ ID NO: 215 - DNA, 20 bp, Homo sapiens
tcacactccagccgtctctt

SEQ ID NO: 216 - DNA, 21 bp, Homo sapiens
gttcctaagagggcatggatg

SEQ ID NO: 217 - DNA, 26 bp, Homo sapiens
ggaaatgaccaaccaccagaaaaaac

SEQ ID NO: 218 - DNA, 24 bp, Homo sapiens
gattagttcacggaccagacatag

SEQ ID NO: 219 - DNA, 22 bp, Homo sapiens
gagagcctactaaaaagcccag

SEQ ID NO: 220 - DNA, 20 bp, Homo sapiens
cgcagaacaccagagagatg

SEQ ID NO: 221 - DNA, 35 bp, Homo sapiens; identical to SEQ ID NO: 85

SEQ ID NO: 222 - DNA, 26 bp, Homo sapiens
cctatgtggttactttgaggttttgg

SEQ ID NO: 223 - DNA, 25 bp, Homo sapiens
gtgtttgctcttgtcagtttcttgg

SEQ ID NO: 224 - DNA, 21 bp, Homo sapiens
cctggcctcctattgttgagt

SEQ ID NO: 225 - DNA, 27 bp, Homo sapiens
gtgtaccaggattcattgtttcacatc

SEQ ID NO: 226 - DNA, 20 bp, Homo sapiens
gttcatcctttccctgccac

SEQ ID NO: 227 - DNA, 21 bp, Homo sapiens
cctgagggaactggcatgata

SEQ ID NO: 228 - DNA, 24 bp, Homo sapiens
cagaatgatgtgactggtggtaac

SEQ ID NO: 229 - DNA, 23 bp, Homo sapiens
cttcagactttacacaacctgcg

SEQ ID NO: 230 - DNA, 25 bp, Homo sapiens
gagtgctgagattgattctgatgac

SEQ ID NO: 231 - DNA, 20 bp, Homo sapiens
gtcttttggggagagtggtc

SEQ ID NO: 232 - DNA, 22 bp, Homo sapiens
cgcaaacgctatcagcaagaag

SEQ ID NO: 233 - DNA, 24 bp, Homo sapiens
ctagagaagcaagcaagaatggtc

SEQ ID NO: 234 - DNA, 20 bp, Homo sapiens
gattgaaggtgcaagccacc

SEQ ID NO: 235 - DNA, 25 bp, Homo sapiens
ggtcctagagaaaaggatgttacag

SEQ ID NO: 236 - DNA, 20 bp, Homo sapiens
gggtcatcaggaagaactgc

SEQ ID NO: 237 - DNA, 22 bp, Homo sapiens
cattctggagttccttgagtgg

SEQ ID NO: 238 - DNA, 22 bp, Homo sapiens
caactgtcctgctctttgaagc

SEQ ID NO: 239 - DNA, 26 bp, Homo sapiens
ctaggttggtagttcttggaatgtag

SEQ ID NO: 240 - DNA, 20 bp, Homo sapiens
ttcctggagaagagaggctg

SEQ ID NO: 241 - DNA, 20 bp, Homo sapiens
agagagaggcggagacctta

SEQ ID NO: 242 - DNA, 25 bp, Homo sapiens
gtttccagactctctttcaatgctg

SEQ ID NO: 243 - DNA, 20 bp, Homo sapiens
ccaacagtgactccgtcaag

SEQ ID NO: 244 - DNA, 21 bp, Homo sapiens
caccccagactgtggctttta

SEQ ID NO: 245 - DNA, 20 bp, Homo sapiens
actgaatgtccccttggctc

SEQ ID NO: 246 - DNA, 20 bp, Homo sapiens
tagaaggtgagcggtcactg

SEQ ID NO: 247 - DNA, 22 bp, Homo sapiens
cacctcatttagttcgtgcgag

SEQ ID NO: 248 - DNA, 20 bp, Homo sapiens
ctgcccggcattattcagag

SEQ ID NO: 249 - DNA, 20 bp, Homo sapiens
ctttcaggagccaggattcc

SEQ ID NO: 250 - DNA, 21 bp, Homo sapiens
ggaccttgaatcccaccttgt

SEQ ID NO: 251 - DNA, 20 bp, Homo sapiens
atcctgccatcaccatccac

SEQ ID NO: 252 - DNA, 33 bp, Homo sapiens
gaaagttctcctctgtgtttgtctctagtttgg

SEQ ID NO: 253 - DNA, 41 bp, Homo sapiens
ccctgccccggttcatcctgatggagctcatggcgggggga

SEQ ID NO: 254 - DNA, 41 bp, Homo sapiens
tccccccgccatgagctccatcaggatgaaccggggcaggg

SEQ ID NO: 255 - DNA, 21 bp, Homo sapiens
ggccctactgccctgtgtgtc

SEQ ID NO: 256 - DNA, 22 bp, Homo sapiens
caactggcagaaaccagcccgt

SEQ ID NO: 257 - DNA, 45 bp, Homo sapiens
tctcgggccatcccgaagtctgcaatcttggccactcttccaggg

SEQ ID NO: 258 - DNA, 45 bp, Homo sapiens
ccctggaagagtggccaagattgcagacttcgggatggcccgaga

SEQ ID NO: 259 - DNA, 27 bp, Homo sapiens
gccacttagaattcctgagtactgagg

SEQ ID NO: 260 - DNA, 16 bp, Homo sapiens
ccgccatgagctccat

SEQ ID NO: 261 - DNA, 22 bp, Homo sapiens
ccaccagaacattgttcgctgc

SEQ ID NO: 262 - DNA, 18 bp, Homo sapiens
  misc_feature (18)..(18)
  misc_feature (18)..(18): 3-phos
gctccagcaggatgaacc

SEQ ID NO: 263 - DNA, 19 bp, Homo sapiens
gaagagtggccaagattgc

SEQ ID NO: 264 - DNA, 16 bp, Homo sapiens
cggaggggtgaggcag

SEQ ID NO: 265 - DNA, 20 bp, Homo sapiens
  misc_feature (20)..(20): 3-phos
gccaagattggagacttcgg